(19) United States
(12) Patent Application Publication
(10) Pub. No.: US 2008/0242615 A1
(43) Pub. Date: Oct. 2, 2008

(54) SURFACTANT PROTEIN-D FOR PREVENTION AND TREATMENT OF LUNG INFECTIONS AND SEPSIS

(75) Inventors: Machiko Ikegami, Cincinnati, OH (US); Jeffrey A. Whitsett, Cincinnati, OH (US)

Correspondence Address: KNOBBE MARTENS OLSON & BEAR LLP, 2040 MAIN STREET, FOURTEENTH FLOOR, IRVINE, CA, 92614, US

(73) Assignee: CHILDREN'S HOSPITAL MEDICAL CENTER, Cincinnati, OH (US)

(21) Appl. No.: 12/111,900

(22) Filed: Apr. 29, 2008

Related U.S. Application Data
(63) Continuation of application No. PCT/US2006/043055, filed on Nov. 3, 2006.
(60) Provisional application No. 60/734,017, filed on Nov. 3, 2005.

(51) Int. Cl. A61K 38/00; A61P 9/00

(52) U.S. Cl. 514/12

(57) ABSTRACT

Surfactant protein D (SP-D) is a member of the collectin family of collagenous lectin domain-containing proteins that is expressed in epithelial cells of the lung. Administration of SP-D protein or fragments thereof is useful for the prevention or treatment of sepsis or lung infection.

### RELATED APPLICATIONS

This application is a continuation under 35 U.S.C. § 365 (c) claiming the benefit of the filing date of PCT Application No. PCT/US2006/043055 designating the United States, filed Nov. 3, 2006. The PCT Application was published in English as WO 2007/056195 on May 18, 2007 and republished in English as WO 2007/056195 on Sep. 27, 2007, and claims the benefit of the earlier filing date of U.S. Provisional Application Ser. No. 60/734017, filed Nov. 3, 2005. The contents of the U.S. Provisional Application Ser. No. 60/734017 and the International Application No. PCT/US2006/043055 including the publication WO 2007/056195 are incorporated herein by reference in their entirety.

### STATEMENT REGARDING FEDERALLY SPONSORED R&D

Certain aspects of the invention disclosed herein were made with United States government support under NIH (National Institutes of Health) Grant No. HL63329. The United States government has certain rights in these aspects of the invention.

### REFERENCE TO SEQUENCE LISTING

The present application is being filed along with a Sequence Listing in electronic format. The Sequence Listing is provided as a file entitled SEQLIST_CHMC31_{—}001C1.TXT, created Apr. 21, 2008, which is 8 Kb in size. The information in the electronic format of the Sequence Listing is incorporated herein by reference in its entirety.

### BACKGROUND OF THE INVENTION

1. Field of the Invention

The invention relates to the field of biologically active proteins and their pharmaceutical use. More specifically, the invention relates to SP-D proteins and their administration to individuals to prevent or treat sepsis.

2. Description of the Related Art

Pulmonary surfactant is essential for normal lung mechanics and gas exchange in the lung. Pulmonary surfactant is produced by type II epithelial cells and is made up of a phospholipid component which confers the ability of surfactant to lower surface tension in the lung. In addition, there are proteins associated with the surfactant called collectins which are collagenous, lectin domain-containing polypeptides. One of these surfactant proteins, termed surfactant protein D (SP-D), is likely to be involved in surfactant structure and function and host defense.

Sepsis is a serious, often life-threatening, disease typically caused by high levels of bacterial endotoxins resulting from an overwhelming bacterial infection in the blood stream. While sepsis can originate from many bodily tissues, such as kidneys, liver, bowel, and skin, it is often derived from an initial infection in the lung.

Individuals of any age can be susceptible to sepsis. Infants are particularly susceptible to sepsis because of the immaturity of their immune system. Low-birth weight infants, for example, (<1500 g) frequently experience serious systemic infections (Kaufman et al., (2004) Clin Microbiol Rev, 17:638-680, which is incorporated herein by reference in its entirety) and septicemia-related shock that are common through exposure to chorioamnionitis in utero and pulmonary infections after birth (Goldenberg et al., (2000) N Engl J Med, 342:1500-1507, Wenstrom et al., (1998) Am J Obstet Gynecol, 178:546-550, each of which is incorporated herein by reference in its entirety). Because of its immaturity, the preterm newborn lung is highly permeable, allowing the leak of proteins, organisms, toxins and mediators from the lung into the systemic circulation (Pringle et al., (1986) Clin Obstet Gynecol, 29:502-513; Jobe et al., (1985) J Appl Physiol, 58:1246-1251; Bland et al., (1989) J Clin Invest, 84:568-576, each of which is incorporated herein by reference in its entirety). Neonatal sepsis syndrome, associated with pneumonia and chorioamnionitis, is a common cause of neonatal morbidity and mortality in both term and preterm infants (Kaufman et al., (2004) Clin Microbiol Rev, 17:638-680, Dempsey et al., (2005) Am J Perinatol, 22:155-159, Jiang et al., (2004) J Microbiol Immunol Infect, 37:301-306, each of which is incorporated herein by reference in its entirety). In previous studies, systemic inflammation was caused by the leak of intratracheal lipopolysaccharides (LPS) into the systemic circulation in premature newborn lambs (Kramer et al., (2002) Am J Respir Crit Care Med, 165:463-469, which is incorporated herein by reference in its entirety).

The susceptibility of neonates to pulmonary and systemic infection has been associated with the immaturity of both their lung structure and immune system. The lungs of preterm infants are deficient in pulmonary surfactant and innate host defense proteins, including surfactant proteins (SP)A and D (Mason et al., (1998) Am J Physiol, 275:L1-L13; Miyamura et al., (1994) Biochim Biophys Acta, 1210:303-307; Awasthi et al., (1999) Am J Respir Crit Care Med, 160:942-94910-12, each of which is incorporated herein by reference in its entirety). Surfactant replacement preparations used for respiratory distress in neonates contain SP-B and SP-C but do not contain SP-A, SP-D or other innate host defense proteins. Pulmonary collectins play an important role in protection of the lung from viral, bacterial and fungal pathogens. Both SP-A and SP-D have anti-microbial and anti-inflammatory activities (Mason et al., (1998) Am J Physiol, 275:L1-L13; Crouch et al., (2001) Annual Review of Physiology, 63:521-554, each of which is incorporated herein by reference in its entirety). Decreased levels of SP-A and SP-D have been associated with lung inflammation in models of bronchopulmonary dysplasia (BPD) (Awasthi, S. et al. (1999) Am J Respir Crit Care Med 160:942-949, which is incorporated herein by reference in its entirety) and in children with cystic fibrosis (Noah et al., (2003) Am J Respir Crit Care Med, 168:685-691; Postle et al., (1999) Am J Respir Cell Mol Biol, 20:90-98; von Bredow et al., (2003) Lung, 181:79-88, each of which is incorporated herein by reference in its entirety) that can influence the pathogenesis of disease and lead to sepsis.

Methods of reducing susceptibility of individuals to sepsis, and methods of treating sepsis, particularly by use of administration of immunity-related proteins that are typically naturally present in the lungs, are useful for treating patients of all ages who are at risk for sepsis.

### SUMMARY OF THE INVENTION

The invention relates generally to methods and compositions containing SP-D or a fragment thereof, or a recombinant form thereof, for the prevention and treatment of lung infection and sepsis in a patient.

In some embodiments of the present invention, a method of preventing or treating sepsis in an individual is provided, by administering a polypeptide having at least 70% homology to an SP-D polypeptide or a fragment thereof to individual. The individual can be, for example, a mammal, and can be a human. The individual can be, for example, an adult, a child, an infant, a newborn, or a premature newborn. The administration can be performed, for example, by intratracheal administration, aerosolization, or systemic administration. The sepsis can be derived, for example, from a bacterial infection or from a lung infection. The polypeptide can be a recombinant polypeptide. The recombinant polypeptide can be, for example, recombinant human surfactant protein D. The polypeptide can be administered, for example, in a range from about 0.50, 1, 2, 5, or 10 mg polypeptide per kg body weight to about 15, 20, 30, 40, 50, or 100 mg polypeptide per kg body weight. The polypeptide can be administered, for example, at about 2 mg polypeptide per kg body weight. The SP-D formulation can be administered, for example, by intratracheal administration, aerosolization, or systemic administration, and can be in a form suitable for intratracheal administration, aerosolization, or systemic administration. The recombinant polypeptide can have an amino acid sequence from about 5 amino acids to about 375 amino acids.

In additional embodiments of the present invention, a method of preventing or treating sepsis in an individual is provided, by administering a nucleic acid encoding a polypeptide having at least 70% homology to an SP-D polypeptide or a fragment thereof to the individual.

In further embodiments of the present invention, a method of decreasing leakage of lipopolysaccharides (LPS) to blood plasma in an individual is provided, by administering a polypeptide having at least 70% homology to an SP-D polypeptide or a fragment thereof to the individual.

In some embodiments of the present invention, a method of decreasing leakage of E. coli cells to blood plasma in an individual is provided, by administering a polypeptide having at least 70% homology to an SP-D polypeptide or a fragment thereof to the individual.

In additional embodiments of the present invention, a method of decreasing endotoxin levels in blood plasma in an individual is provided, by administering a polypeptide having at least 70% homology to an SP-D polypeptide or a fragment thereof to the individual.

In some embodiments of the present invention, a method of inhibiting the release of endotoxins from the lung is provided, by administering a polypeptide having at least 70% homology to an SP-D polypeptide or a fragment thereof.

In further embodiments of the present invention, a method of protecting individuals from systemic effects of intratracheal endotoxin is provided, by administering a polypeptide having at least 70% homology to an SP-D polypeptide or a fragment thereof to the individual.

In additional embodiments of the present invention, a method of preventing systemic inflammation is provided, by administering a polypeptide having at least 70% homology to an SP-D polypeptide or a fragment thereof to the individual. The systemic inflammation can be, for example, caused by release of endotoxins from the lung.

In yet further embodiments of the present invention, a method for treating an individual with a lung infection is provided, by administering SP-D or a fragment thereof. The lung infection can be, for example, caused by a bacterium.

In some embodiments of the present invention, a method for treating an individual with a lung infection is provided, so that the risk of sepsis is decreased, by administering SP-D or a fragment thereof.

In some embodiments of the present invention, a pharmaceutical composition including an SP-D polypeptide or an active fragment thereof is provided. The SP-D polypeptide in the pharmaceutical composition can be, for example, a recombinant SP-D polypeptide. The recombinant SP-D polypeptide can be, for example, a recombinant human SP-D polypeptide. The SP-D polypeptide can include, for example, the sequence listed in SEQ ID NO: 2 or SEQ ID NO: 3. Furthermore, the pharmaceutical composition including the SP-D polypeptide can, for example, additionally include a pharmaceutically acceptable dispersing agent. The pharmaceutical composition can be formulated, for example, for intratracheal administration, aerosolization, or systemic administration. The pharmaceutical composition can also be formulated such that the SP-D polypeptide is administered, for example, in a range from about 0.50, 1, 2, 5, or 10 mg polypeptide per kg body weight to about 15, 20, 30, 40, 50, or 100 mg polypeptide per kg body weight. The pharmaceutical composition can be formulated such that the SP-D polypeptide is administered, for example, at about 2 mg polypeptide per kg body weight.

In other embodiments of the present invention, a pharmaceutical composition containing a nucleic acid encoding an SP-D polypeptide or an active fragment thereof is provided. The nucleic acid can include, for example, the sequence listed in SEQ ID NO: 1. The nucleic acid can also, for example, be encoded within an adenoviral vector.

### BRIEF DESCRIPTION OF THE DRAWINGS

FIG. 1 is a Kaplan-Meier plot comparing the rhSP-D treated group and control group. In the control group, only 20% of the lambs survived before the end of the 5 h study period. In contrast, all lambs treated with rhSP-D survived. p<0.05 by log-rank test.

FIG. 2A is a line graph comparison of plasma endotoxin levels in rhSP-D-treated vs. the untreated control group. Intratracheal endotoxin was detected in circulation and was increased over time in control group, while rhSP-D decreased plasma endotoxin concentration during the 5 h of study.

FIG. 2B is a line graph comparing the systolic blood pressure measurement in rhSP-D-treated vs. the untreated control group. Treatment with rhSP-D prevented the endotoxin shock. Systolic blood pressure was maintained at normal level of premature newborn in rhSP-D treated groups. In contrast, blood pressure was gradually decreased in the control group after 3 h of age. *p<0.05 vs. control.

FIG. 3A is a line graph comparing blood pH in the rhSP-D-treated vs. the untreated control group. Blood pH was maintained with rhSP-D treatment. While LPS treatment associated with decreased blood pH, treatment with rhSP-D maintained pH and prevented prenatal endotoxin induced shock.

FIG. 3B is a line graph comparing BE (Blood Base Excess) in the rhSP-D-treated vs. the untreated control group. BE was altered by intratracheal LPS. Intratracheal LPS induced metabolic acidosis and rhSP-D treatment prevented the low BE and endotoxin shock.

FIG. 4 demonstrates the sequential measurement of pCO₂ and ventilatory pressure. FIG. 4A is a line graph comparing pCO₂ in the rhSP-D-treated vs. the untreated control group. Endotracheal LPS caused an increase in pCO₂ after 3 h of age. pCO₂ was maintained when treated with rhSP-D.

FIG. 4B is a line graph comparing ventilatory pressure (PIP-PEEP) in the rhSP-D-treated vs. the untreated control group. The amount of ventilatory pressure used to maintain target tidal volume was similar for both groups. *p<0.05 vs. control.

FIG. 5 is a comparison of pro-inflammatory cytokine expression in the rhSP-D-treated vs. the untreated control group. FIGS. 5A and 5B are bar graphs demonstrating that pro-inflammatory cytokines IL-1β, IL-6 and IL-8 mRNAs in spleen and liver increased in control lambs after intratracheal LPS instillation. Pro-inflammatory cytokine mRNAs in spleen and liver were decreased by rhSP-D administration.

FIG. 5C is a bar graph demonstrating that Endotracheal LPS increased IL-1β, Il-6 and IL-8 mRNAs in the lung. Expression of IL-1β decreased when treated with rhSP-D.

FIG. 5D is a line graph showing IL-8 concentrations in plasma. The plasma IL-8 levels were increased in the control group. Plasma IL-8 concentrations maintained a low level by rhSP-D treatment. *p<0.05 vs. control.

FIG. 6 includes several histological images showing lung morphology with hematoxylin and eosin staining (6A and 6B) and immunohistochemistry of IL-8 (6C and 6D) and IL-1β (6E and 6F). In both control and rhSP-D groups there are increased granulocyte and positively stained inflammatory cells for IL-8 and IL-1β. The inflammatory cells immunostained for IL-8 and IL-1β was decreased by intratracheal rhSP-D treatment.

FIGS. 7A and 7B are line graphs demonstrating that lung function was not affected by rhSP-D treatment. FIG. 7A shows the dynamic lung compliance, calculated from VT, PIP-PEEP and body weight during ventilation. FIG. 7B demonstrates that the deflation limb of static lung pressure volume curve measurements were similar between the control and rhSP-D groups.

FIG. 8 is an immunoblot demonstrating that high levels of rhSP-D were detected in bronchoalveolar lavage fluid (BALF) five hours after endotracheal rhSP-D instillation (Animals #6, 7 and 8). rhSP-D was not found in BALF from control lambs (animals #1 and 2).

FIG. 9 demonstrates that SP-D significantly decreased IL-6 and TNFα levels in the plasma in a concentration dependent manner when administered with LPS. FIG. 9A shows the IL-6 data, and FIG. 9B shows the TNFα data.

FIG. 10 shows that SP-D lowered plasma IL-6 levels when administered before (t=−30), with (t=0), or after (t=+30) the LPS dose compared to plasma IL-6 levels in the absence of SP-D treatment.

FIG. 11 shows that inhibition of LPS-induced inflammation directly correlated with SP-D LPS binding affinity. FIG. 11A illustrates the LPS binding affinity of two separate E. coli strains for SP-D. Strain 011:B4 has high SP-D LPS binding affinity, whereas strain 0127:B8 has low SP-D LPS binding affinity. FIG. 11B demonstrates that pre-incubating the high binding LPS strain (strain 011:B4) with SP-D significantly decreased plasma IL-6 levels; however, SP-D did not inhibit inflammation induced by the LPS strain with low affinity for SP-D (strain 0127:B8).

FIG. 12 is a comparison of plasma cytokine levels in wild type and Sftpd^{-/-} mice following systemic LPS exposure. Plasma IL-6 levels in Sftpd^{-/-} mice treated with LPS were about 80% lower than in wild type mice, which was an unexpected result.

FIG. 13 is a comparison of plasma cytokine levels in systemically septic mice treated with and without SP-D. Following cecal ligation and puncture (CLP), mice treated with SP-D exhibited lower mean plasma IL-6 levels than control mice.

FIG. 14 is a comparison of survival in systemically septic mice treated with and without SP-D. Following CLP, mortality was significantly higher in control mice than in mice treated with SP-D.

FIG. 15 is a comparison of plasma SP-D levels in septic and control mice. Plasma SP-D levels increased significantly in sepsis-induced mice relative to those in control mice, indicating that the mouse CLP model can provide a functional in vivo system to evaluate systemic SP-D production.

FIG. 16 demonstrates that the Sftpd promoter is activated in vascular endothelial cells. MFLM-91U cells, an immortalized mouse fetal lung mesenchyme cell line, were transiently transfected with a plasmid containing the Sftpd promoter coupled to a luciferase reporter gene or with a control plasmid containing the luciferase reporter gene alone. Luciferase activity was significantly increased in MFLM-91U cells transfected with the plasmid containing the Sftpd promoter coupled to the luciferase gene compared to control plasmid-transfected cells.

FIG. 17 is a line graph showing plasma SP-D levels over time in wild type and Sftpd^{-/-} mice. SP-D remained in the plasma with a half life of about 6 hours in wild type mice, but in Sftpd^{-/-} mice, SP-D half life decreased to approximately 2 hours. Interestingly, the half life of a truncated SP-D fragment consisting of a trimer of only the neck and carbohydrate recognition domain (CRD) is 62 hours (Sorensen, G. L. et al., (2006), Am J Physiol Heart Circ Physiol 290: H2286-H2294). Taken together, the results indicate that a specific cellular mechanism for uptake of plasma SP-D exists and that this mechanism is dependent on the N-terminus and/or collagen domain of SP-D.

FIG. 18 illustrates SP-D levels in tissue homogenates in Sftpd^{-/-} mice after administration of SP-D via tail vein injection. Levels of SP-D in the spleen were significantly higher than SP-D levels observed in the other tissues and against background signal in the spleen, indicating that systemic SP-D is cleared from the circulation by the spleen.

FIG. 19 illustrates pulmonary morphology and macrophage activity in wild type and Sftpd^{-/-} mice in which a mutant transgene, rSftpdCDM^{Tg+}, was expressed. The mutant transgene rSftpdCDM^{Tg+} expresses a mutant SP-D protein, rSftpdCDM, that has a normal CRD, neck domain and N-terminal domain but lacks the collagen domain. The mutant SP-D protein did not disrupt pulmonary morphology or macrophage activity in wild type mice; however, it failed to rescue the abnormal baseline macrophage activity of Sftpd^{-/-} mice. Enlarged foamy macrophages that expressed increased levels of metalloproteinases were readily observed in Sftpd^{-/-} mice and Sftpd^{-/-} mice that expressed the rSftpdCDM protein. FIG. 19A illustrates lung tissue from wild type mice. FIG. 19B illustrates expression of the rSftpdCDM^{Tg+} transgene in a wild type background. FIG. 19C shows lung tissue from Sftpd^{-/-} mice. FIG. 19D shows expression of the rSftpdCDM^{Tg+} transgene in Sftpd^{-/-} background. Arrowheads in the figures point to enlarged, foamy macrophages.

FIG. 20 illustrates the responses of wild type, Sftpd^{-/-}, and rSftpdCDM^{Tg+}/Sftpd^{-/-} mice to intratracheal exposure to influenza A virus (IAV). Increased levels of IL-6, TNFα and IFNγ were observed in the lung homogenates of IAV-challenged Sftpd^{-/-} mice. However, these levels were restored to wild-type levels in the lung homogenates rSftpdCDM^{Tg+}/Sftpd^{-/-} mice. FIGS. 20A shows data for plasma IL-6 levels in the three groups of IAV-challenged mice. FIGS. 20B and 20C likewise illustrate results for plasma TNFα levels and IFNγ levels, respectively, in the three groups of IAV-challenged mice.

FIG. 21 is a schematic representation of available Sftpd promoter constructs that are used in experiments to identify regions of the Sftpd promoter that are important for expression in vascular endothelial cells.

### DETAILED DESCRIPTION OF THE PREFERRED EMBODIMENT

The lung is constantly challenged by inhaled particles and microorganisms, yet it remains remarkably healthy. This is due in large part to the pulmonary collecting, surfactant protein A (SP-A) and surfactant protein D (SP-D) (Kingma, P. S., and J. A. Whitsett, (2006) Curr Opin Pharmacol, 6:277-83; Crouch, E. and J. R. Wright, (2001) Annu Rev Physiol 63:521-54; Hawgood, S. and F. R. Poulain, (2001) Annu Rev Physiol 63:495-519; Whitsett, J. A., (2005) Biol Neonate 88:175-80, each of which is incorporated herein by reference in its entirety). SP-D recognizes and binds infectious organisms via interactions between the SP-D carbohydrate recognition domain and carbohydrate moieties on the organism's surface, which in turn facilitates clearance of the infectious pathogens by alveolar macrophages (Kishore, U. et al., (1996) Biochem J 318:505-511; Lim, B. L. et al., (1994) Biochem Biophys Res Commun 202:1674-80; Kuan, S. F. et al., (1992) J Clin Invest 90:97-106, each of which is incorporated herein by reference in its entirety. Mice with targeted deletion of the SP-D gene (Sftpd^{-/-}) develop gradually worsening pulmonary emphysema and inflammation indicating that in addition to binding infectious particles, SP-D can have important roles in regulating pulmonary host defense cells (Korfhagen, T. R. et al., (1998) J Biol Chem 273:28438-29443; Wert, S. E. et al., (2000) Proc Natl Acad Sci USA 97:5972-7; Clark, H. et al., (2002) J Immunol 169:2892-2899, each of which is incorporated herein by reference in its entirety). As a consequence of its role in the lung immune system, SP-D is being developed as a therapeutic agent designed to limit the growth of microorganisms in the lung and the resulting inflammatory damage. In addition to the respiratory tree, SP-D is also detected in lower concentrations in plasma and many other non-pulmonary tissues, including vascular endothelium (Stahlman, M. T. et al., (2002) J Histochem Cytochem 50:651-60; Honda, Y. et al., (1995) Am J Respir Crit Care Med 152:1860-6; Sorensen, G. L. et al., (2006) Am J Physiol Lung Cell Mol Physiol 290: L1010-L1017; Sorensen, G. L. et al., (2006), Am J Physiol Heart Circ Physiol 290: H2286-H2294, each of which is incorporated herein by reference in its entirety). Extrapulmonary levels of SP-D increase during infection and other proinflammatory states in a manner similar to intrapulmonary SP-D (Sorensen, G. L. et al., (2006) Am J Physiol Lung Cell Mol Physiol 290: L110-L1017; Fujita, M. et al., (2005) Cytokine 31:25-33, each of which is incorporated herein by reference in its entirety); however the source and functions of extrapulmonary SP-D are largely unknown. As herein described, preliminary studies show that SP-D is also involved in host defense beyond the pulmonary system and can clear infectious pathogens and regulate host defense cells in extrapulmonary systems.

SP-D is a multimeric glycoprotein of the collectin family of innate immune molecules, and is secreted by airway epithelial cells. SP-D binds to and aggregates a wide range of microbial pathogens, including bacteria, viruses, fungi, and mite extracts (Kuan et al., (1992) J Clin Invest, 90:97-106; Lim et al., (1994) Biochem Biophys Res Commun, 202:1674-1680; van Rozendaal et al., (1999) Biochim Biophys Acta, 1454:261-269; Hartshorn et al., (1996) Am J Physiol Lung Cell Mol Physiol, 271:L753-L762, each of which is incorporated herein by reference in its entirety), and directly binds to bacterial components such as LPS, peptidoglycan and lipoteichoic acid (Crouch et al., (2001) Annual Review of Physiology, 63:521-554; van de Wetering, J. K. et al., (2004) Eur J Biochem 271:1229-1249, each of which is incorporated herein by reference in its entirety). The multimeric form of SP-D allows SP-D to bind ligands on the surface of different microorganisms thereby forming protein bridges between microbes that induce microbial aggregation and stimulate immune cell mediated recognition and clearance of pulmonary pathogens (Hartshorn, K. et al., (1996) Am J Physiol 271:L75362; Hartshorn, K. L. et al., (1998) Am J Physiol 274:L958-L969, each of which is incorporated herein by reference in its entirety). By interacting with these microbes or microbial components, SP-D limits inflammation induced by pulmonary infection or LPS by inhibiting activation of alveolar macrophages. (Kuan et al., (1992) J Clin Invest, 90:97-106; Van Rozendaal, B. A. et al., (1997) Biochem Soc Trans 25:S656; van Rozendaal, B. A. et al., (1999) Biochim Biophys Acta 1454:261-9; Schaub, B. et al., (2004) Clin Exp Allergy 34:1819-26; Liu, C. F. et al., (2005) Clin Exp Allergy 35:515-521, each of which is incorporated herein by reference in its entirety).

Most microbial ligands contain mannose or glucose and SP-D is known to bind preferentially to inositol, maltose, mannose and glucose. Unlike SP-A, SP-D does not bind to the lipid A domain (Van Iwaarden et al., (1994) Biochem J, 303 (Pt 2):407-411, which is incorporated herein by reference in its entirety) but binds to the contiguous core oligosaccharide of LPS (Crouch et al., (1998) Am J Respir Cell Mol Biol, 19:177-201; Crouch et al., (1998) Biochim Biophys Acta, 1408:278-289, each of which is incorporated herein by reference in its entirety). Furthermore, the maximum molecular dimension of SP-D is 5-fold greater than SP-A and SP-D has greater binding surfaces than SP-A (Crouch et al., (1998) Am J Respir Cell Mol Biol, 19:177-201, which is incorporated herein by reference in its entirety).

SP-D binds to the surface of Escherichia via its C-terminal lectin-like domain. Further, the binding of SP-D to pathogens promotes the killing of pathogens by pulmonary phagocytes (Mason et al., (1998) Am J Physiol, 275:L1-L13; Crouch et al., (2001) Annual Review of Physiology, 63:521-554; Kuan et al., (1992) J Clin Invest, 90:97-106; Lim et al., (1994) Biochem Biophys Res Commun, 202:1674-1680; van Rozendaal et al., (1999) Biochim Biophys Acta, 1454:261-269; Crouch et al., (1998) Am J Respir Cell Mol Biol, 19:177-201, each of which is incorporated herein by reference in its entirety). Mice lacking SP-D (Sftpd^{-/-} mice) are highly susceptible to pulmonary infection and inflammation (LeVine et al., (2004) Am J Respir Cell Mol Biol, 31:193-199; LeVine et al., (2001) J Immunol, 167:5868-5873, each of which is incorporated herein by reference in its entirety).

### SP-D Regulates Alveolar Macrophages

Although binding infectious organisms is a key feature of SP-D physiology, mouse models of SP-D deficiency revealed more complex roles of this protein in pulmonary host defense. Mice with deletion of the Sftpd gene survived normally, but had elevated surfactant lipid pool sizes and spontaneously developed pulmonary inflammation and airspace enlargement (Korlhagen, T. R. et al., (1998) J Biol Chem 273:28438-29443; Wert, S. E. et al., (2000) Proc Natl Acad Sci USA 97:5972-7; Clark, H. et al., (2002) J Immunol 169:2892-2899). Baseline alveolar macrophage activity is elevated in Sftpd^{-/-} mice as evident by increased numbers of apoptotic macrophages and enlarged, foamy macrophages that released reactive oxygen species and metalloproteinases (MMP). Uptake and clearance of viral pathogens including influenza A and respiratory syncytial virus were deficient in Sftpd^{-/-} mice (LeVine et al., (2004) Am J Respir Cell Mol Biol, 31:193-199; LeVine et al., (2001) J Immunol, 167:5868-5873). In contrast, clearance of group B Streptococcus and Haemophilus influenza was unchanged (LeVine, A. M. et al., (2000) J Immunol 165:3934-3940, which is incorporated herein by reference in its entirety). However, oxygen radical release and production of the proinflammatory mediators TNFα, IL-1, and IL-6 were increased in Sftpd^{-/-} mice when exposed to either viral or bacterial pathogens, indicating that SP-D also plays an important role in regulating alveolar macrophages during infectious challenge that is independent of the clearance of pathogens (LeVine et al., (2004) Am J Respir Cell Mol Biol, 31:193-199; LeVine et al., (2001) J Immunol, 167:5868-5873; LeVine, A. M. et al., (2000) J Immunol 165:3934-3940).

In the lung, SP-D is produced by alveolar type II and other nonciliated bronchiolar epithelial cells and cleared by alveolar macrophages and type II cells (Crouch, E. et al., (1992) Am J Physiol 263:L60-L66; Voorhout, W. F. et al., (1992) J Histochem Cytochem 40:1589-97; Crouch, E. et al., (1991) Am J Respir Cell Mol Biol 5:13-18; Dong, Q. and J. R. Wright, (1998) J. R. Am J Physiol 274:L97-105; Herbein, J. F. et al., (2000) Am J Physiol Lung Cell Mol Physiol 278:L830-L839; Kuan, S. F. et al., (1994) Am J Respir Cell Mol Biol 10:430-436, each of which is incorporated herein by reference in its entirety). The source of extrapulmonary SP-D and the mechanisms that control SP-D levels in plasma has hitherto been unknown. SP-D present in plasma can be produced outside the lung, and control of systemic levels of SP-D can occur through either activation of systemic expression pathways or by changing systemic SP-D clearance.

SP-D has been implicated in several immune cell signaling pathways. SP-D binds the LPS receptor CD14 via interactions between the carbohydrate recognition domain (CRD) and N-linked oligosaccharides on CD14 (Sano, H. et al., (2000) J Biol Chem 275:22442-22451, which is incorporated herein by reference in its entirety). SP-D also inhibits interactions between CD14 and both smooth and rough forms of LPS (Sano, H. et al., (2000) J Biol Chem 275:22442-51). In addition, CD14 receptor levels are decreased on alveolar macrophages from Sftpd^{-/-} mice, whereas soluble CD14 levels are increased (Senft, A. P. et al., (2005) J Immunol 174:4953-4959, which is incorporated herein by reference in its entirety). Soluble CD14 levels returned to wild type levels in Sftpd^{-/-} mice with targeted deletion of the MMP-9 or -12 genes, suggesting that SP-D controls CD14 receptor levels by inhibiting MMP-9 or -12 mediated proteolytic cleavage of the receptor (Senft, A. P. et al., (2005) J Immunol 174:4953-4959).

SP-D binds the extracellular domains of toll-like receptors (TLR)-2 and -4, which are involved in initiating the inflammatory response to LPS, peptidoglycan, and lipoteichoic acid (Ohya, M. et al., (2006) Biochemistry 45:8657-8664, which is incorporated herein by reference in its entirety). Whereas SP-A inhibits TLR-2 activation by peptidoglycan (Sato, M. et al., (2003) J Immunol 171: 417-25; Murakami, S. et al., (2002) J Biol Chem 277:6830-7, each of which is incorporated herein by reference in its entirety), the effect of SP-D on TLR-2 or -4 signaling is currently unknown.

Gardai et al. proposed a model by which SP-D might simultaneously mediate anti- and pro-inflammatory processes in the lung through the opposing actions of signal regulating protein α (SIRPα) and calreticulin/CD91 (Gardai, S. J. et al., (2003) Cell 115:13-23, which is incorporated herein by reference in its entirety). Their model indicates that in the unbound state, the CRD of SP-D inhibits macrophage activation by binding to SIRPα which inhibits P38 mediated activation of NFκB. In contrast, if the CRD of SP-D is occupied by a microbial ligand, binding to SIRPα is inhibited and the collectin binds to the macrophage activating receptor, calreticulin/CD91. Calreticulin/CD91 subsequently stimulates P38 mediated activation of NFκB which induces pro-inflammatory mediators and activates alveolar macrophages. Therefore, depending on the presence or absence of infectious particles in the CRD and type of receptor bound, SP-D can either enhance or suppress inflammation.

SP-D influences NFκB activity through oxidant sensitive pathways (Yoshida, M. et al., (2001) J Immunol 166:7514-9, which is incorporated herein by reference in its entirety). Alveolar macrophages from Sftpd^{-/-} mice produce increased amounts of hydrogen peroxide. The increase in reactive oxygen species in Sftp^{-/-} mice was associated with an increase in markers of oxidative stress, including tissue lipid peroxides and reactive carbonyls, which in turn activated NFκB and increased MMP production.

SP-D also influences MHC class II presentation of bacterial antigens and subsequent T-cell activation (Hansen, S. et al., (2006) Am J Respir Cell Mol Biol, which is incorporated herein by reference in its entirety). Interestingly, SP-D enhanced antigen presentation by bone marrow derived dendritic cells, whereas antigen presentation by pulmonary dendritic cells was inhibited. These results indicate that the effect of SP-D on systemic host defense cells and the signaling pathways regulated by systemic SP-D can diverge from those observed in the lung.

### Expression of SP-D

SP-D is encoded by a single gene (Sftpd) located in close proximity to the SP-A gene on human chromosome 10 (Crouch, E. et al., (1993) J BioI Chem 268:2976-83, which is incorporated herein by reference in its entirety). Although SP-D was first recognized in the lung and is expressed primarily by type II and other non-ciliated bronchiolar respiratory epithelial cells (Crouch, E. et al., (1992) Am J Physiol 263:L60-L66; Voorhout, W. F. et al., (1992) J Histochem Cytochem 40:1589-97; Crouch, E. et al., (1991) Am J Respir Cell Mol Biol 5:13-18; Dong, Q. and J. R. Wright, (1998) J. R. Am J Physiol 274:L97-105; Herbein, J. F. et al., (2000) Am J Physiol Lung Cell Mol Physiol 278:L830-L839; Kuan, S. F. et al., (1994) Am J Respir Cell Mol Biol 10:430-436), SP-D mRNA and protein are detected in many non-pulmonary tissues. SP-D immunostaining is detected in vascular endothelium and the epithelial cells of parotid glands, sweat glands, lachrymal glands, skin, gall bladder, bile ducts, pancreas, stomach, esophagus, small intestine, kidney, adrenal cortex, anterior pituitary, endocervical glands, seminal vesicles, and urinary tract (Stahlman, M. T. et al., (2002) J Histochem Cytochem 50:651-660; Sorensen, G. L. et al., (2006) Am J Physiol Lung Cell Mol Physiol 290: L1010-L1017; Fisher, J. H. and R. Mason, (1995) Am J Respir Cell Mol Biol 12:13-18; Motwani, M. et al., (1995) J Immunol 155:5671-5677, each of which is incorporated herein by reference in its entirety). Extrapulmonary levels of SP-D mRNA increase in response to inflammation, but they are several-fold lower than mRNA levels detected in the lung indicating that different mechanisms control extrapulmonary versus intrapulmonary Sftpd expression (Sorensen, G. L. et al., (2006) Am J Physiol Lung Cell Mol Physiol 290: L1010-L1017).

SP-D mRNA is first detected in the mouse or rat lung at midgestation and increases prior to birth and during the neonatal period (Crouch, E. et al., (1991) Am J Respir Cell Mol Biol 5:13-18). SP-D mRNA increases following lung injury caused by bacterial endotoxin, inhaled microorganisms, and hyperoxia (Cao, Y. et al., (2004) J Allergy Clin Immunol 113: 439-444; Mcintosh, J. C. et al., (1996) Am J Respir Cell Mol Biol 15:509-519; Jain-Vora, S. et al., (1998) Infect Immun 66:4229-4236; Aderibigbe, A. O. et al., (1999) Am J Respir Cell Mol Biol 20: 219-227, each of which is incorporated herein by reference in its entirety). The mouse Sftpd promoter contains consensus transcription factor binding sequences for the AP-1 family, forkhead transcription factors FoxA1 and FoxA2, thyroid transcription factor (TTF)-1, nuclear factor of activated T cells (NFAT), and multiple sites for CCAAT enhancer binding proteins (C/EBP's) (Lawson, P. R. et al., (1999) Am J Respir Cell Mol Biol 20: 953-963, which is incorporated herein by reference in its entirety). The AP-1 family member proteins JunB and JunD enhanced Sftpd promoter activity, whereas c-Jun and c-Fos inhibited Sftpd transcription (He, Y. et al., (2000) J Biol Chem 275:31051-31060, which is incorporated herein by reference in its entirety). Deletion of the FoxA1 and FoxA2 consensus binding sites inhibited transcription (He, Y. et al., (2000) J Biol Chem 275:31051-31060). C/EBP's activate the transcription of Sftpd (He, Y. et al., (2000) J Biol Chem 275:31051-31060; Gotoh, T. et al., (1997) J Biol Chem 272: 3694-3698, each of which is incorporated herein by reference in its entirety). C/EBP's are also involved in the systemic acute phase response, which indicates that systemic SP-D expression can be part of the physiologic response to systemic infection. NFAT also promotes Sftpd promoter activity through calcineurin dependent pathways and direct interaction with TTF-1 (Dave, V. et al., (2004) J Biol Chem 279: 34578-34588, which is incorporated herein by reference in its entirety).

### Role of SP-D in Non-Pulmonary Tissues

Because of the relatively low concentration of SP-D in non-pulmonary tissues, investigations of the physiological role and therapeutic potential of SP-D have been largely limited to the respiratory tree. SP-D is present at low levels in human plasma and multiple studies have demonstrated an increase in plasma SP-D during infection and/or exposure to pulmonary toxicants (Honda, Y. et al., (1995) Am J Respir Crit Care Med 152:1860-6; Kuroki, Y. et al., (1998) Biochim Biophys Acta 1408: 334-345; Greene, K. E. et al., (2002) Eur Respir J 19: 439-46; Greene, K. E. et al., (1999) Am J Respir Crit Care Med 160:1843-1850, each of which is incorporated herein by reference in its entirety). This increase has been interpreted to represent leakage of SP-D from the lung, and several groups are currently developing methods to use plasma SP-D levels as a clinical biomarker of lung injury. However, many of the agents used to induce pulmonary injury and inflammation in these studies also induce systemic injury and inflammation. Therefore, the relative contribution of pulmonary versus systemic sources to plasma SP-D pool sizes is unknown.

SP-D present in the amniotic fluid and the female reproductive tract can protect against intrauterine infection (Oberley, R. E. et al., (2004) Mol Hum Reprod 10:861-870; Leth-Larsen, R. et al., (2004) Mol Hum Reprod 10:149-154, each of which is incorporated herein by reference in its entirety). SP-D is present in tears and inhibits invasion of corneal epithelial cells by Pseudomonas aeruginosa (Ni, M. et al., (2005) Infect Immun 73:2147-2156, which is incorporated herein by reference in its entirety). Although these findings indicate a physiologic purpose for extrapulmonary SP-D, the ability of plasma SP-D to regulate systemic host defense cells or to bind and facilitate the clearance of systemic pathogens has yet to be determined.

### Clinical Applications of SP-D

In the lung, SP-D has both pro- and anti-inflammatory properties which promote a controlled response by alveolar macrophages to pulmonary infection that simultaneously facilitates the clearance of invading pathogens while maintaining the delicate integrity of the lung parenchyma. The anti-inflammatory properties of SP-D indicate that this protein can limit damage from persistent inflammation associated with asthma, bronchopulmonary dysplasia, cystic fibrosis, adult respiratory distress syndrome, or chronic infection. In support of this indication, administration of SP-D or a truncated form of SP-D reduces the allergic response in mice suffering from allergic airway hypersensitivity (Liu, C. F. et al., (2005) Clin Exp Allergy 35:515-521; Haczku, A. et al., (2004) Clin Exp Allergy 34: 1815-1818; Kasper, M. et al., (2002) Clin Exp Allergy 32:1251-1258, each of which is incorporated herein by reference in its entirety).

Although SP-D deficiency is associated with prematurity and artificial surfactant replacement therapies are widely used in premature infants with respiratory distress syndrome (clinical trials of surfactant therapy in other pulmonary diseases are ongoing), SP-D is not a component of artificial surfactant. Mouse models clearly demonstrate that deficiencies of SP-D result in increased susceptibility to pulmonary infection (LeVine et al., (2004) Am J Respir Cell Mol Biol, 31:193-199; LeVine et al., (2001) J Immunol, 167:5868-5873; LeVine, A. M. et al., (2000) J Immunol 165:3934-3940). Restoring SP-D in Sftpd^{-/-} mice reverses defects in pulmonary microbial clearance and inflammation (Zhang, L. et al., (2002) J Biol Chem 277:38709-38713; LeVine, A. M. et al., (1999) Am J Respir Cell Mol Biol 20:279-286, each of which is incorporated herein by reference in its entirety). In addition, intratracheally-administered recombinant SP-D markedly improves survival and decreases systemic release of LPS in premature newborn sheep exposed to intratracheal LPS and ventilator-induced lung injury (Ikegami, M. et al., (2006) Am J Respir Crit Care Med, which is incorporated herein by reference in its entirety). Taken together, these studies reveal the potential value of SP-D as an antimicrobial agent during pulmonary infection in patients with immune defects or surfactant protein deficiencies. Considering that levels of pulmonary SP-D increase as part of the physiological response to infection, supplementing this process with exogenous SP-D during the early stages of infection can also benefit patients with intact immune systems.

In the lung, SP-D is involved in facilitating clearance of invading pathogens and limiting the damaging effects of LPS induced inflammation. However, infections outside the pulmonary system induce some of the most clinically significant morbidity and mortality. Infants with congenital or perinatally acquired pneumonia are at high risk of splenic sepsis and death, even when effective antibiotic treatment is given soon after birth (Kaufman et al., (2004) Clin Microbiol Rev, 17:638-680; Goldenberg et al., (2000) N Engl J Med, 342:1500-1507; Wenstrom et al., (1998) Am J Obstet Gynecol, 178:546-550; Dempsey et al., (2005) Am J Perinatol, 22:155-159, each of which is incorporated herein by reference in its entirety). The high incidence of congenital pneumonia in early onset sepsis indicates that infection is often acquired by aspiration of pathogens in utero or during birth. Chorioaniionitis increases the risk of premature delivery and is strongly associated with neonatal sepsis and septicemia related shock (Dempsey et al., (2005) Am J Perinatol, 22:155-159, which is incorporated herein by reference in its entirety). The preterm newborn lung is highly permeable (Jobe et al., (1985) J Appl Physiol, 58:1246-1251, which is incorporated herein by reference in its entirety) allowing systemic spread of pro-inflammatory mediators and organisms from the lung (Kramer et al., (2002) Am J Respir Crit Care Med, 165:463-469, which is incorporated herein by reference in its entirety). In premature infants alone, about 20% of infants weighing less than 1500 grams at birth will be diagnosed with a systemic infection before discharge from the hospital (Stoll, B. J. et al., (2002) Pediatrics 110:285-291; Brodie, S. B. et al., (2000) Pediatr Infect Dis J 19:56-65, each of which is incorporated herein by reference in its entirety). The majority of these infants will develop sepsis, the clinical signs and symptoms of the host derived inflammatory response to infection (Bone, R. C., (1996) Jama 276:565-566; Angus, D. C. et al., (2001) Crit Care Med 29:1303-1310; Glauser, M. P. et al., (1991) Lancet 338:732-736, each of which is incorporated herein by reference in its entirety). Ultimately, of the approximately 20% of premature infants diagnosed with infection, 18% will die from sepsis (Stoll, B. J. et al., (2002) Pediatrics 110:285-291; Brodie, S. B. et al., (2000) Pediatr Infect Dis J 19:56-65).

Group B streptococcus and gram-negative bacteria including E. coli are organisms commonly causing congenital pneumonia (Stoll et al., (2005) Pediatr Infect Dis J. 24:635-639, which is incorporated herein by reference in its entirety). Systemic spread of microbial toxins and LPS, rather than bacteria itself, can initiate the cellular and humoral responses resulting in shock (Grandel et al., (2003) Crit Rev Immunol, 23:267-299, which is incorporated herein by reference in its entirety). Septic shock is a complex pathophysiologic state which often leads to multiple organ dysfimction, multiple organ failure and death (Murphy et al., (1998) New Horiz, 6:181-193, which is incorporated herein by reference in its entirety). Decreases in blood pH, blood base excess (BE) and increases in pCO₂, demonstrated in the control group in the present study, are typical of the clinical course of septic shock in premature infants. Vasoconstriction, pulmonary hypertension, deterioration of organ circulation and metabolic acidosis frequently implicates the presence of sepsis. In the examples illustrated below, we show that SP-D can be an important component of the systemic innate immune system and determine the physiological function of SP-D in systemic host defense to assess the therapeutic potential of SP-D in treating systemic infection.

Treatment with SP-D

Exogenously prepared SP-D can be useful for treating diseases such as lung infections that can eventually lead to systemic sepsis if unchecked. To determine whether SP-D administration can reduce the risk of sepsis in an individual, preterm newborn lambs were instilled with E. coli-derived lipopolysaccharide endotoxins, and were then treated with SP-D as described herein. Survival rate, physiological lung function, lung and systemic inflammation and endotoxin level in plasma were then evaluated. As shown herein, intratracheal recombinant human Surfactant Protein-D (rhSP-D) prevented shock caused by endotoxin released from the lung during ventilation in the premature newborn. In addition, transgenic mouse lines lacking the SP-D gene or expressing a doxycycline-inducible lung specific SP-D transgene or expressing SP-D mutant transgenes were developed to allow structure/function studies of the protein. As shown herein, administration of SP-D inhibits inflammation induced by systemic LPS and reduces inflammation in cecal ligation and puncture. In addition, administration of SP-D improves survival and tissue injury after the administration of lethal doses of LPS, increases clearance rates of plasma LPS, and prevents systemic and pulmonary leaks of LPS. Accordingly, SP-D treatment can be useful to treat or prevent sepsis.

### Results of Experimental Studies in Lambs

Recombinant human Surfactant Protein-D (rhSP-D) was synthesized by transfection of CHO DHFR cells with a cDNA encoding full length human SP-D as described in Example 1. SP-D was isolated from the culture medium using ion exchange chromatography and affinity purification as described in Example 1.

Biologically active recombinant human and rat SP-D have been previously produced in vitro (Erpenbeck et al., (2005) Am J Physiol Lung Cell Mol Physiol, 288:L692-698; Clark et al., (2002) J Immunol, 169:2892-2899; Clark et al., (2002) Immunobiology, 205:619-631, each of which is incorporated herein by reference in its entirety). Full-length recombinant SP-D was utilized in this study. A dose of 2 mg/kg rhSP-D was given to the premature lamb. The 130 d GA lamb (term 150 d) is surfactant deficient (Docimo et al., (1991) Anat Rec, 229:495-498; Ikegami et al., (1981) Am J Obstet Gynecol, 141:227-229, each of which is incorporated herein by reference in its entirety) and requires surfactant treatment and mechanical ventilation to survive. Surfactant pool sizes change with age and are highest in newborn animals (Ikegami et al., (1993) Semin Perinatol, 17:233-240, which is incorporated herein by reference in its entirety) and decrease with advancing age to adult levels (Ikegami et al., (2000) Am J Physiol Lung Cell Mol Physiol, 279:L468-L476, which is incorporated herein by reference in its entirety). The clinical dose of surfactant for treatment is similar to the surfactant pool size in the normal newborn (Ikegami et al., (1980) Pediatr Res, 14:1082-1085, which is incorporated herein by reference in its entirety). The precise amount of SP-D in the term newborn lung is unknown. SP-D in near-term (175 d GA) baboon (term—185 d GA) was 0.02 mg/lung in bronchoalveolar lavage fluid (BALF) and 0.2 mg/lung in lung tissue (Awasthi et al., (1999) Am J Respir Crit Care Med, 160:942-949, which is incorporated herein by reference in its entirety). Since a near-term baboon weighs less than 1 kg, the dose of rhSP-D used in the present study (2 mg/kg) was estimated to be at least 10-fold higher than the SP-D pool size for the term newborn lamb.

To prepare the animals for treatment, preterm lambs were delivered by Cesarean section at 130 d gestation age as described in Example 2. An endotracheal tube was tied into the trachea, and excess fetal lung fluid was removed. To facilitate uniform distribution of lipopolysaccharide (LPS) in the lung, 0.1 mg/kg E. coli-derived LPS was mixed with 1 ml (25 mg) Survanta and administered to the lambs before the first breath, followed by 10 ml of air, as detailed in Example 3.

Lambs were then treated with either Survanta alone (control group), or Survanta plus rhSP-D (treatment group) as described in Example 4. Animals were ventilated for 5 hours while being carefully monitored as described in Example 4. Five hours after treatment, each animal was deeply anesthetized with 25 mg/kg pentobarbital intravenously and ventilated briefly with 100% oxygen, as described in Example 4.

Methods of analysis of the lamb tissue are described in Examples 5 through 12. Example 5 details the method of preparation of the lamb tissue for processing and sample analysis. Example 6 details the data analysis methods that were used. Example 7 describes method used for processing the lung tissue.

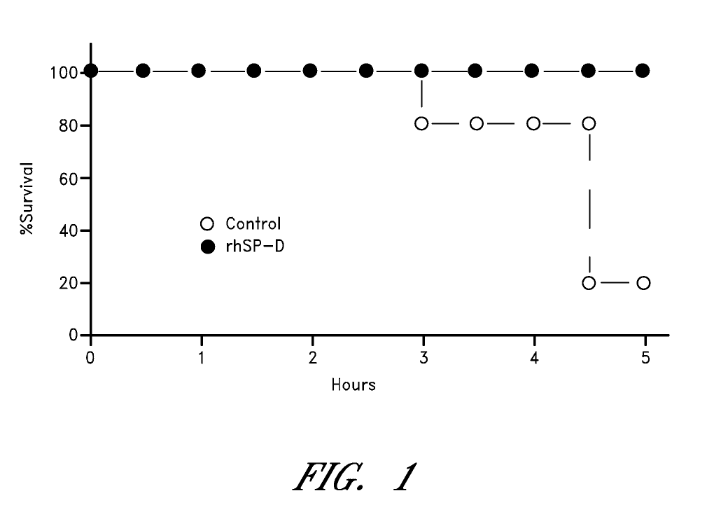

The administration of rhSP-D was found to protect neonatal lambs from systemic effects of intratracheal endotoxin. Five lambs were studied in each group. Body weight (control 3.2±0.3 kg, rhSP-D 3.0±0.2 kg), cord pH (control 7.33±0.02, rhSP-D 7.31±0.04) and sex (3 females and 2 males in both groups) were equally distributed between treated and control groups. In the control group, 4 of 5 lambs died before the end of the 5 h study period. In contrast, all lambs treated with rhSP-D survived (FIG. 1). When the animals died, the data obtained immediately prior to death were used for comparison among the groups. Most deaths in the control group occurred between 4 to 5 h.

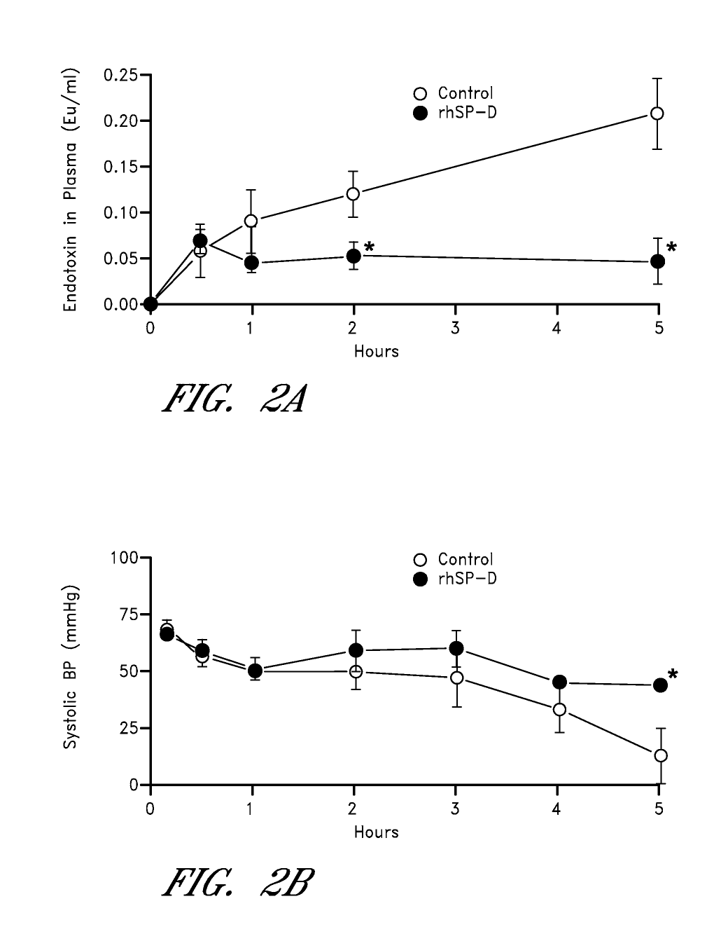

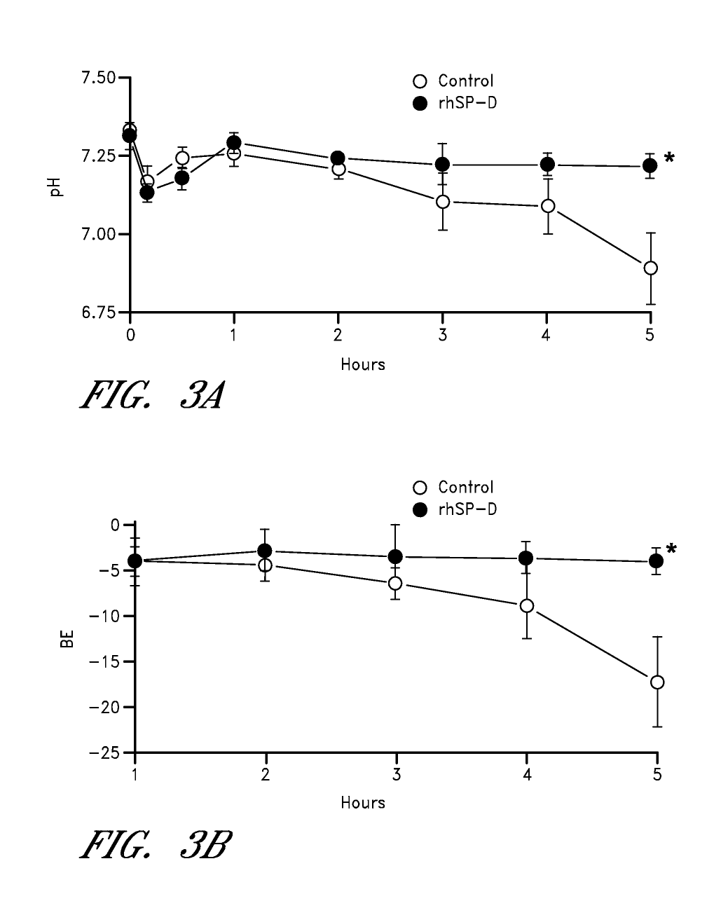

After intratracheal administration, endotoxin was detected in the plasma at 30 min of age in both groups of animals as assessed by Limulus lysate assay (FIG. 2A). Plasma endotoxin levels continued to increase in the control lambs but did not increase over the duration of the experiment in the lambs that were treated with rhSP-D. Systolic blood pressures preceding death were similar between groups at 3 h of age, and decreased thereafter in controls, but not in rhSP-D treated animals (FIG. 2B).

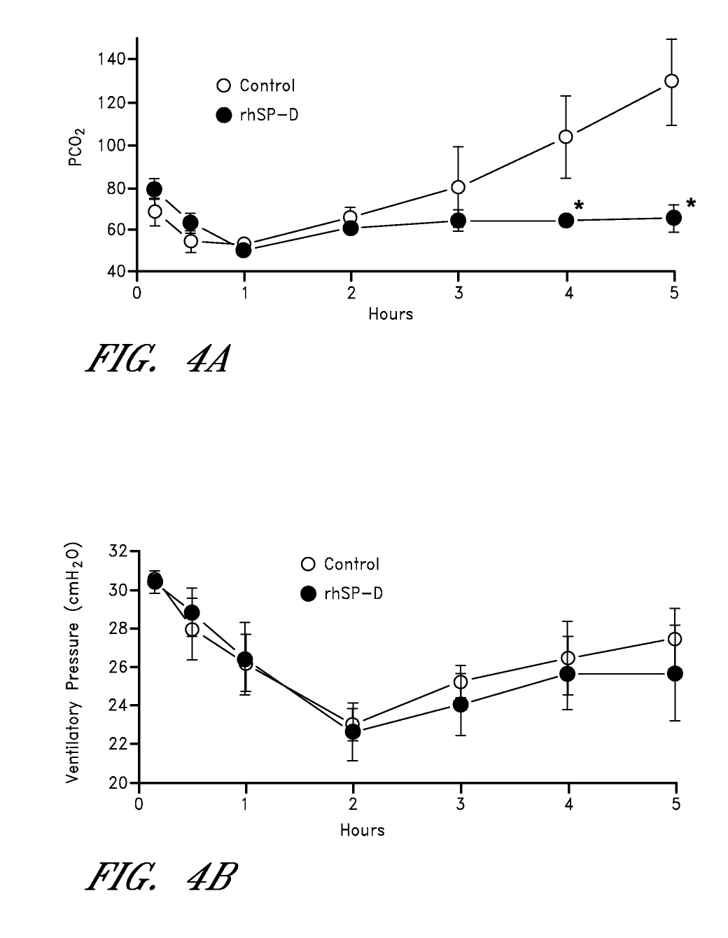

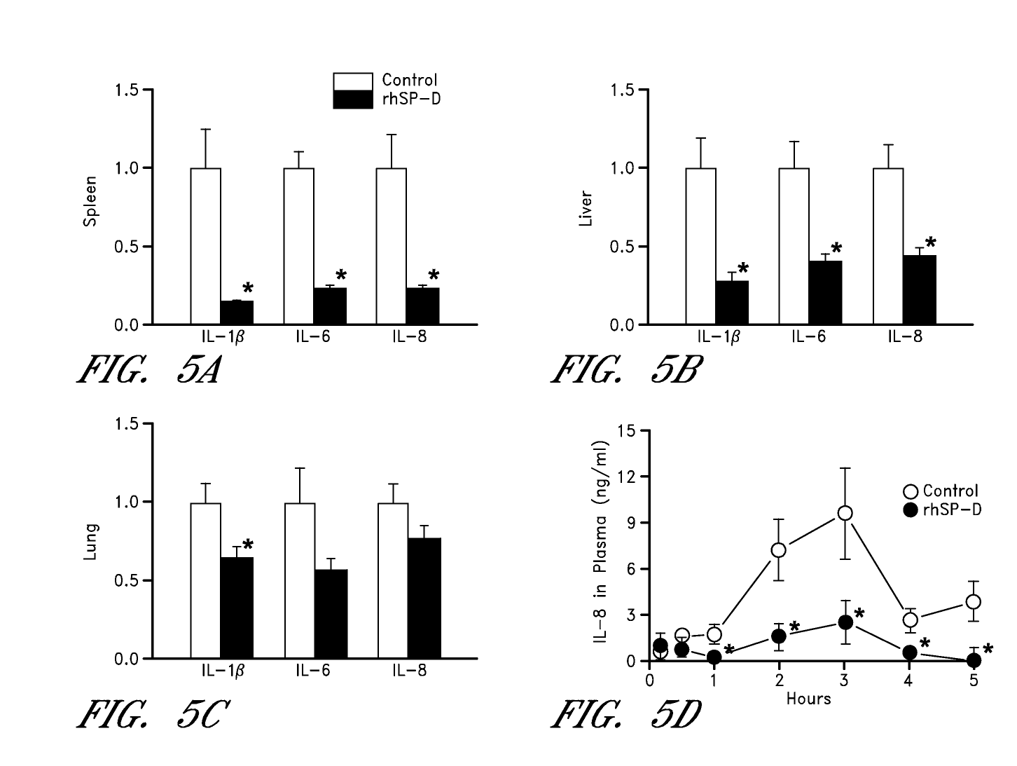

Marked systemic effects of LPS were seen after 4 h of age in the control group as indicated by decreased blood pH, blood base excess (BE) (FIG. 3) and increased PCO₂ (FIG. 4A). In contrast, blood pH, BE and pCO2 remained stable throughout the 5 h of experimentation in the rhSP-D treated animals. Hematocrit, potassium, calcium and glucose levels were similar for both groups. PO₂ was relatively unstable at this gestational age, likely related to patent ductus arteriosis, and was not different between the groups (data not shown).

The method of isolating alveolar cells from the BALF fluid is described in Example 8. The method of measuring the levels of rhSP-D in lung homogenate after centrifugation (BALF) and in serum is described in Example 9. Histology methods used are described in Example 10. Endotoxin levels and cytokine levels were measured as described in Example 11. RNA analysis was performed as described in Example 12.

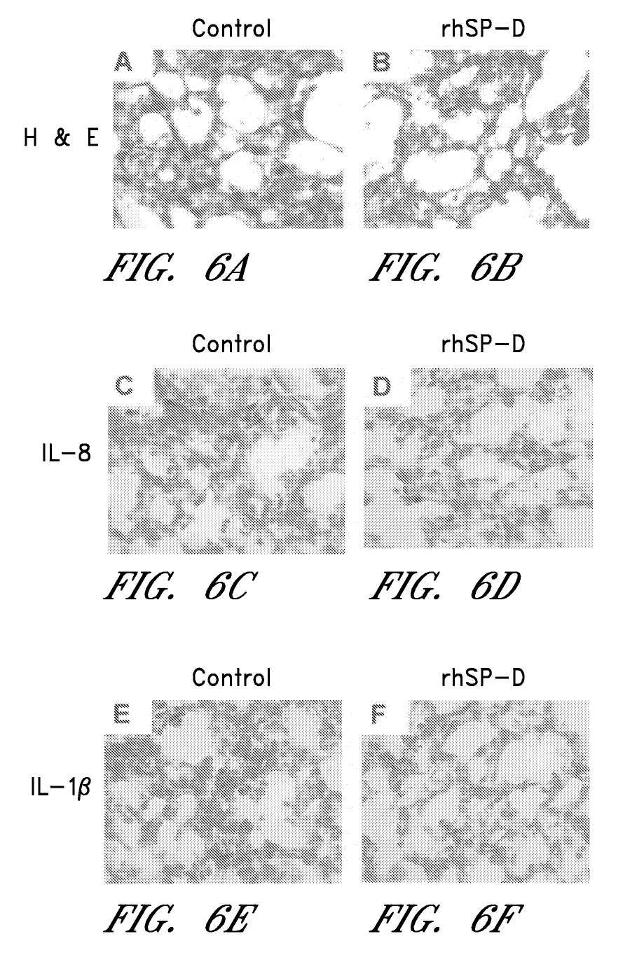

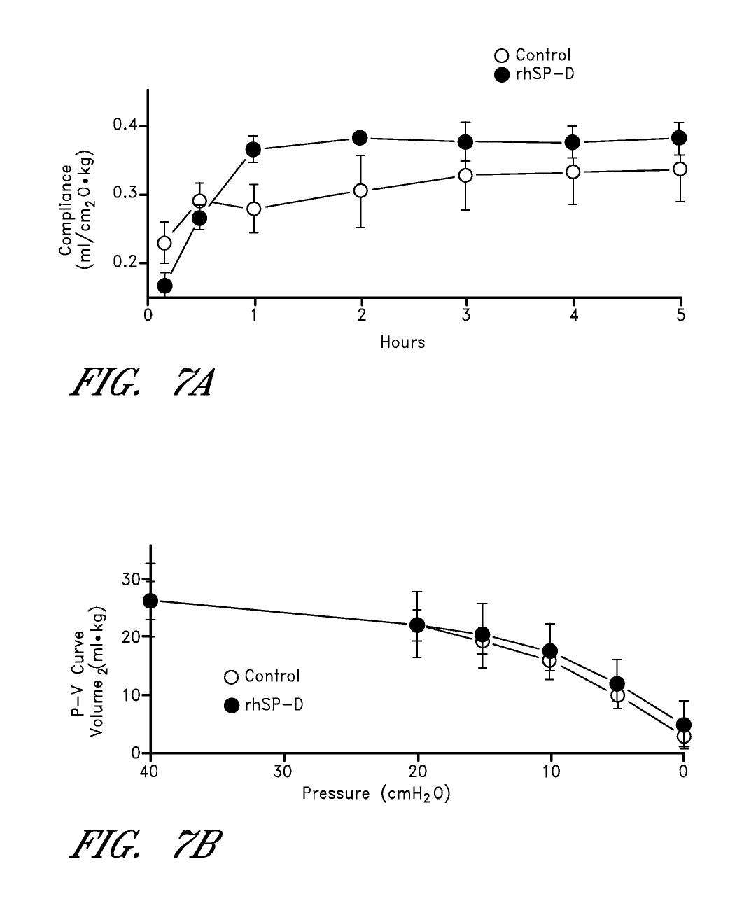

The levels of pro-inflammatory cytokine mRNAs IL-10, IL-6 and IL-8 were increased in the spleen and liver of control animals as compared to the rhSP-D-treated animals. This indicates leakage of LPS from the lungs to the systemic circulation in the absence of rhSP-D (FIGS. 5A and 5B). Splenic and liver levels of IL-10 and TNFα mRNAs were low in both groups of animals (data not shown). Plasma IL-8 was significantly increased in the control group following intratracheal LPS and was significantly lower in rhSP-D treated sheep (FIG. 5D). Plasma IL-1β was below the levels of detectability of the assay (<0.8 pg/ml) in both groups of sheep (data not shown).

Table 1, below, shows the WBC, inflammatory cells, and total protein in BALF. Neutrophil numbers in BALF were similar for both groups, but were 10-fold higher than previously shown for control animals that did not receive LPS (Kramer, B. W. et al. (2002) Am J Respir Crit Care Med 165:463-469, which is incorporated herein by reference in its entirety). Hydrogen peroxide and total protein in BALF were not different between the two groups. The percent apoptotic cells and percent necrotic cells were also similar in both groups (Table 1). Consistent with the anti-inflammatory effect of rhSP-D, pro-inflammatory cytokine IL-1β mRNA was significantly decreased in the lungs of animals treated with rhSP-D (FIG. 5C). rhSP-D reduced the levels of IL-1β in the supernatants of lung homogenates from 21.6±3.6 ng/ml in controls to 12.6±1.4 ng/ml after treatment with rhSP-D (p<0.05). Likewise, rhSP-D decreased IL-6 from 7.7±0.8 ng/ml to 2.3±1.2 ng/ml (p<0.05). IL-8 was not detectable by ELISA in either control or rhSP-D treated groups. Pulmonary inflammation was observed in both rhSP-D treated and control animals (FIG. 5A,B). FIG. 6 illustrates several histological images showing lung morphology with hematoxylin and eosin staining (6A and 6B) and immunohistochemistry of IL-8 (6C and 6D) and IL-1β (6E and 6F). Increased immunostaining for IL-8 (FIG. 6C and 6D) and IL-1β (FIG. 6E and 6F) was observed in both groups of animals, but an increased extent and intensity of staining for both cytokines was observed in the control group, indicating that intratracheal rhSP-D treatment decreased cytokine IL-8 and IL-1β levels in inflammatory cells.

TABLE 1
WBC, Inflammatory Cells and Total Protein in BALF

| | | BALF | | | | |
|---|---|---|---|---|---|---|
| | WBC/ | Cells/ | H₂O₂/ | Apoptotic | | Protein |
| | μl × 10² | μl × 10² | 10⁶ Cell | % | Necrotic % | mg/kg |
| Control | 27 ± 4 | 66 ± 20 | 16 ± 7 | 30 ± 8 | 0.7 ± 0.1 | 67 ± 12 |
| rhSP-D | 30 ± 6 | 96 ± 21 | 8 ± 3 | 35 ± 1 | 0.7 ± 0.2 | 65 ± 12 |

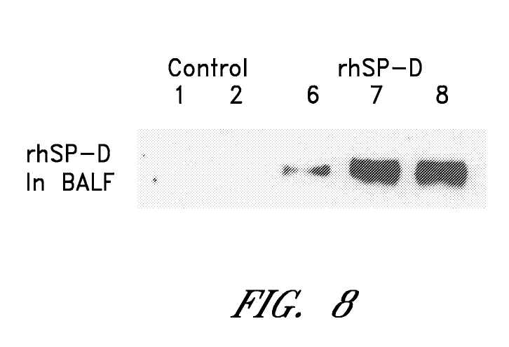

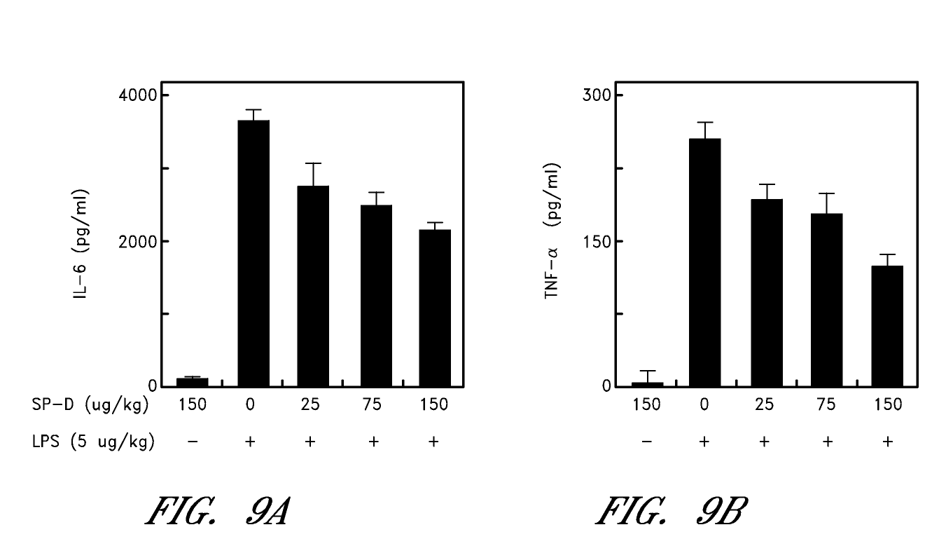

The administration of rhSP-D did not alter pulmonary mechanics following endotoxin exposure. The ventilatory pressure used to maintain target tidal volume was similar in both groups (FIG. 4B). Likewise dynamic lung compliance and pressure-volume curves, as shown in FIG. 7, were not altered by rhSP-D treatment.

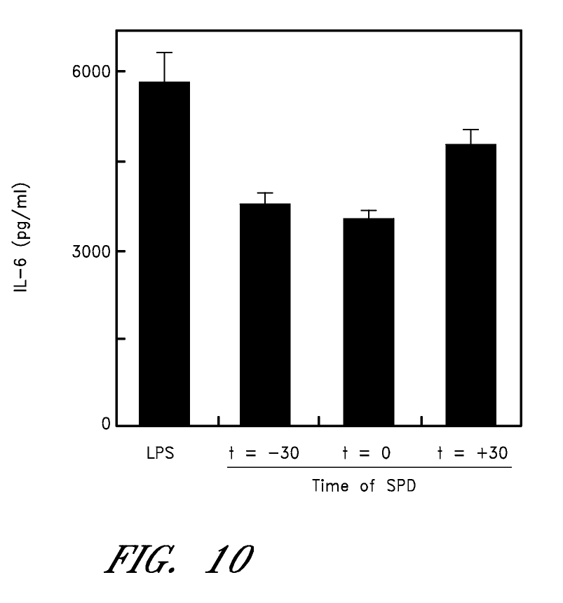

Levels of rhSP-D in BALF, lung homogenate, and plasma were measured at 5 hours after intratracheal administration in both groups by ELISA (Table 2, below) and by an immunoblot in BALF (FIG. 8). The presence of rhSP-D was demonstrated in BALF, lung homogenate and plasma from the rhSP-D group but not the control group. The presence of rhSP-D in the plasma demonstrates its leakage from the lung.

TABLE 2
rhSP-D Level (ng/ml) at 5 h after Treatment

| | BALF | Lung Homogenate | Plasma |
|---|---|---|---|
| Control | 0 | 0 | 0 |
| rhSP-D | 120 ± 33 | 91 ± 25 | 34 ± 7 |

As shown herein, the administration of intratracheal rhSP-D was capable of protecting premature newborn lambs from the systemic effects of intrapulmonary E. coli LPS. While pulmonary inflammation was not blocked by rhSP-D, the systemic effects of LPS, as indicated by levels of LPS in plasma and evidence of systemic inflammation, were ameliorated by rhSP-D. Previous studies demonstrated that systemic inflammation caused by intratracheal LPS in the lamb was age dependent being observed at 130 d GA but not at 141 d GA (Kramer, B. W. et al. (2002) Am J Respir Crit Care Med 165:463-469).

### Mouse Studies: Effect of SP-D on Pulmonary and Systemic Inflammation and Infection

To determine if SP-D limits inflammation induced by systemic LPS, a C57BL/6 wild type mouse model was utilized. Non-lethal doses of E. coli 0111:B4 LPS were administered via tail vein injection with or without stoichiometric amounts of purified recombinant human SP-D (n=5 for each treatment group). LPS (5 μg/kg) was administered with control buffer or increasing concentrations of recombinant human SP-D and the cytokine response was measured in the plasma 2 hours after injection. SP-D significantly decreased levels of IL-6 and TNFα in a concentration dependent manner with 150 μg/kg SP-D producing a maximum reduction of 40% and 50% in IL-6 and TNFα levels, respectively (p<0.01 for each) (FIG. 9).

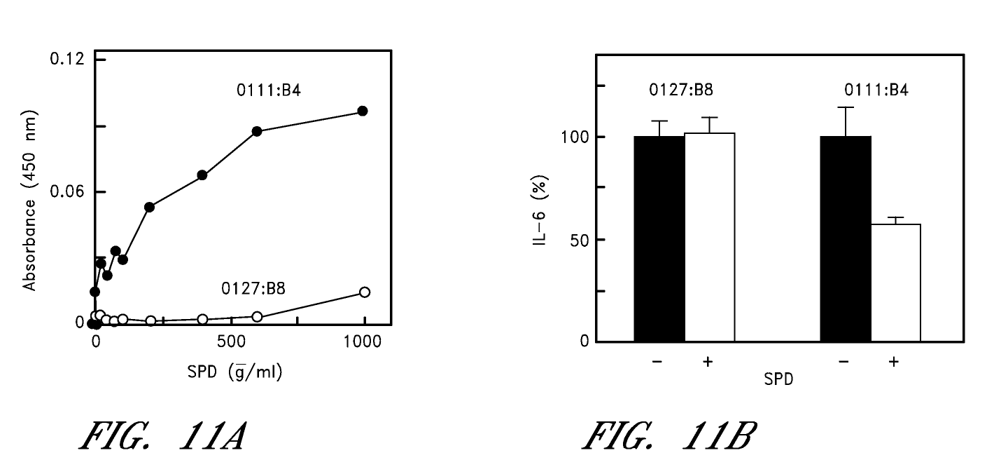

Because LPS was pre-incubated with SP-D prior to injection, this experiment represented optimum conditions for evaluating the effect of SP-D on LPS-induced systemic inflammation. To assess the potential of systemic SP-D to locate and inhibit LPS circulating in the blood, SP-D was administered via tail vein injection 30 minutes before or after LPS injection and the cytokine response was measured in plasma 2 hours later (n=5 mice in each group) (FIG. 10). Systemic IL-6 levels were significantly reduced when SP-D was administered 30 minutes before (p<0.01) or with (p<0.01) LPS injection. IL-6 levels were also lower when SP-D was administered 30 minutes after LPS, but the results did not reach statistical significance (p=0.09). Taken together, the above results indicate that circulating SP-D can inhibit inflammation induced by systemic LPS and that a physiological purpose of increasing systemic SP-D levels during infection is to scavenge systemic LPS and limit the damaging effects of LPS-induced inflammation.

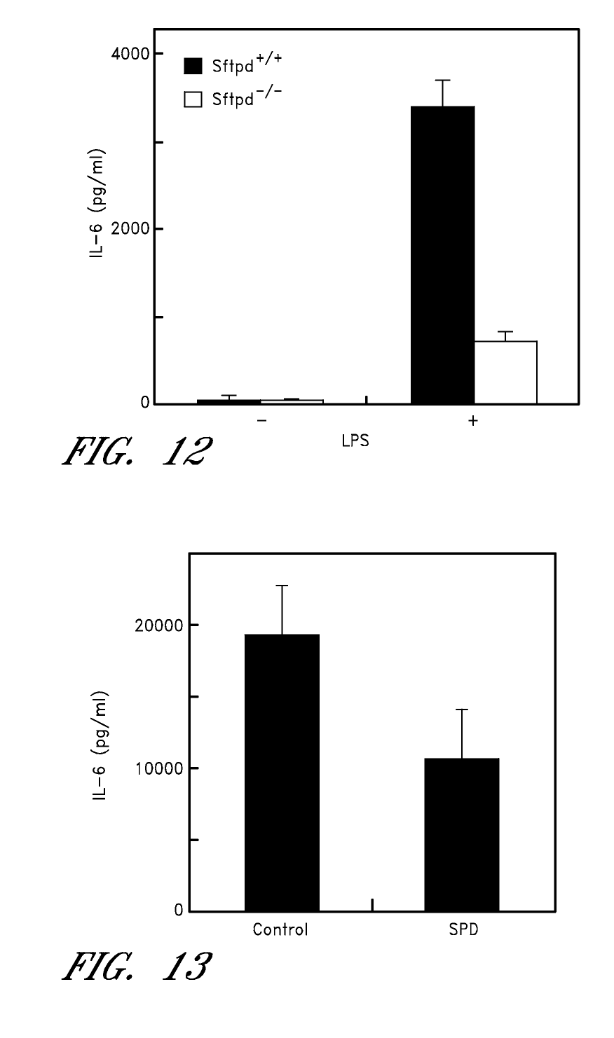

In vitro studies indicate that SP-D can influence several steps in LPS signaling pathways including direct LPS binding, CD14 inhibition, and TLR 4 binding (Sano, H. et al., (2000) J Biol Chem 275:22442-22451; Senft, A. P. et al., (2005) J Immunol 174:4953-4959; Ohya, M. et al., (2006) Biochemistry 45:8657-8664; Gardai, S. J. et al., (2003) Cell 115:13-23). SP-D has a high affinity for the core oligosaccharides of LPS, but the relative affinity varies depending on the strain of bacterial LPS utilized. In contrast, SP-D binding of CD14 and TLR 4 occurs independently of SP-D LPS interactions. Therefore, to determine if SP-D inhibits LPS-induced systemic inflammation through pathways that are dependent or independent of LPS binding, the effect of SP-D on inflammation induced by a low and high SP-D affinity LPS serotype was compared. Using an ELISA-based SP-D LPS binding assay, the binding affinity of SP-D for LPS from several E. coli strains was measured. One strain with a high binding affinity (E. coli 0111:B4) and one with a low binding affinity (E. coli 0127:B8) was identified (FIG. 11A). The effect of SP-D on systemic IL-6 levels 2 hours following tail vein injection of either the low or high binding LPS was determined (n=5 mice in each group). Pre-incubating the high binding LPS with SP-D significantly reduced plasma IL-6 levels, but SP-D did not inhibit inflammation induced by the LPS strain with low affinity for SP-D (FIG. 11B). Therefore, inhibition of LPS-induced inflammation directly correlates with SP-D LPS binding affinity and indicates that systemic SP-D can inhibit LPS-induced inflammation primarily by direct LPS interaction. In addition, the correlation between SP-D LPS binding and SP-D-mediated inhibition of LPS-induced inflammation indicates that the inhibition of LPS observed in these studies is not due to the anti-inflammatory properties of a contaminant within the SP-D preparations.

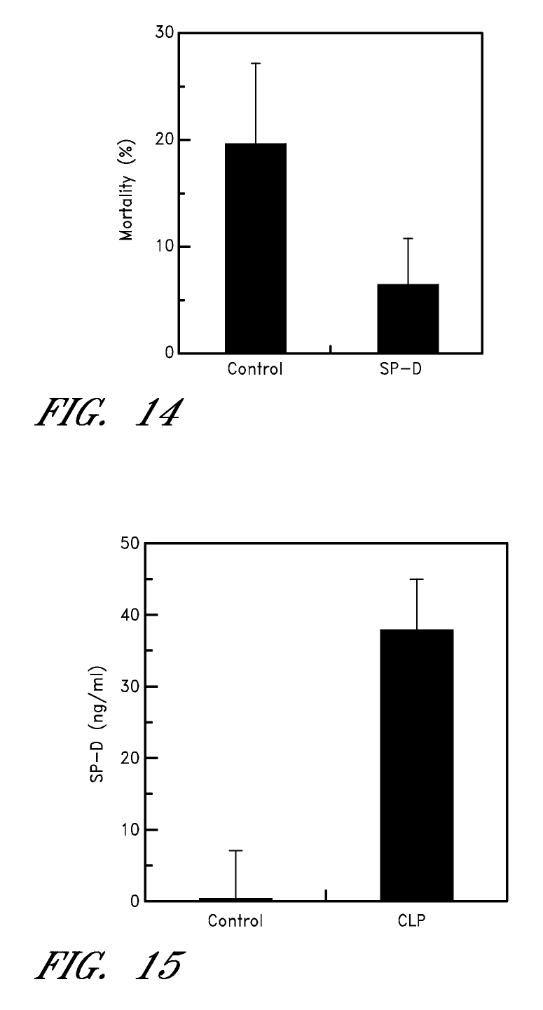

Sftpd mice are characterized by increased pulmonary inflammation at baseline and during infectious challenge (Korfhagen, T. R. et al., (1998) J Biol Chem 273:28438-29443; Wert, S. E. et al., (2000) Proc Natl Acad Sci USA 97:5972-7; Clark, H. et al., (2002) J Immunol 169:2892-2899; LeVine et al., (2004) Am J Respir Cell Mol Biol, 31:193-199; LeVine et al., (2001) J Immunol, 167:5868-5873). Considering the results that SP-D inhibits inflammation induced by systemic LPS and the predominant pro-inflammatory phenotype of Sftpd^{-/-} mice, it was hypothesized that plasma cytokine levels would be elevated in Sftpd^{-/-} mice following systemic LPS exposure. Therefore, both Sftpd^{-/-} and wild type mice (littermate controls) were treated with intravenous LPS, and plasma IL-6 levels were measured 2 hours after injection. In sharp contrast to the elevated pulmonary inflammatory cytokines that are characteristic of Sftpd^{-/-} mice, plasma IL-6 levels in Sftpd^{-/-} mice treated with LPS were approximately 80% lower than wild type mice (FIG. 12). Since SP-D restricts systemic release of pulmonary LPS in sheep subjected to ventilator induced lung injury (Ikegami, M. et al., (2006) Am J Respir Crit Care), the simplest explanation for this surprising result is that Sftpd^{-/-} mice are exposed to a persistent leak of pulmonary LPS into the systemic circulation and subsequently develop LPS tolerance. However, this result can also indicate that SP-D plays an important and complex role in the systemic immune system.

In addition to binding and clearing LPS from the lung, SP-D is an important component of the innate immune response to viral, bacterial, and fungal infections (LeVine et al., (2004) Am J Respir Cell Mol Biol, 31:193-199; LeVine et al., (2001) J Immunol, 167:5868-5873). In vitro studies demonstrate that SP-D binds and aggregates bacteria and viruses and that this aggregation facilitates phagocytosis and killing of infectious organisms by alveolar macrophages (Hartshorn, K. et al., (1996) Am J Physiol 271:L75362; Hartshorn, K. L. et al., (1998) Am J Physiol 274:L958-L969). Systemic SP-D can bind and facilitate the clearance of systemic bacteria which would ultimately lead to less inflammatory tissue damage and improved survival. To investigate this, a clinically relevant mouse model of cecal ligation and puncture (CLP), which induces systemic polymicrobial sepsis/peritonitis, was utilized. Following ligation and puncture of the cecum with a 21-gauge needle by personnel blinded to treatment modality (i.e. SP-D versus control), mice were treated with control buffer or 2 mg/kg SP-D (n=10, 6-8 week old C57/BL6 mice in each group) given by intraperitoneal injection, blood was harvested 6 hours after the procedure, and plasma IL-6 levels were measured. Mice treated with SP-D had mean plasma IL-6 levels that were approximately 40% lower than control mice (FIG. 13). Due to variability within this experiment, these results were not statistically significant (p=0.06), but the trend indicates that SP-D can reduce inflammation during live bacterial challenge.

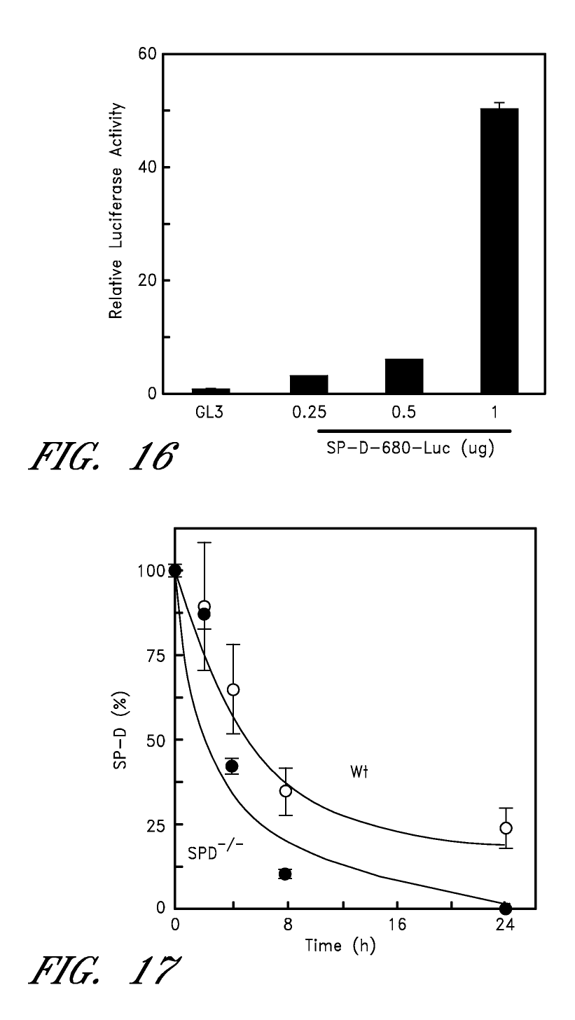

Because of the severity of the sepsis induced by a cecal puncture, a portion of the mice die before the harvest time point (either 6 or 24 hours). As a preliminary study on the effect of SP-D on survival of mice subjected to CLP, the mortality rate following CLP for control mice versus mice treated with SP-D was determined. For the purpose of this experiment, mortality was defined as death before the harvest time point (FIG. 14). Mortality was about 3-fold higher in control mice than in mice treated with SP-D. Since these data are derived from experiments that used a range of cecal puncture sizes, harvest time points, and SP-D doses and routes of administration, the physiological and statistical significance of these results are limited. However, these results indicate that systemic SP-D can decrease inflammation and improve survival of mice during live bacterial challenge.

### Mouse Studies: Expression and Clearance of SP-D

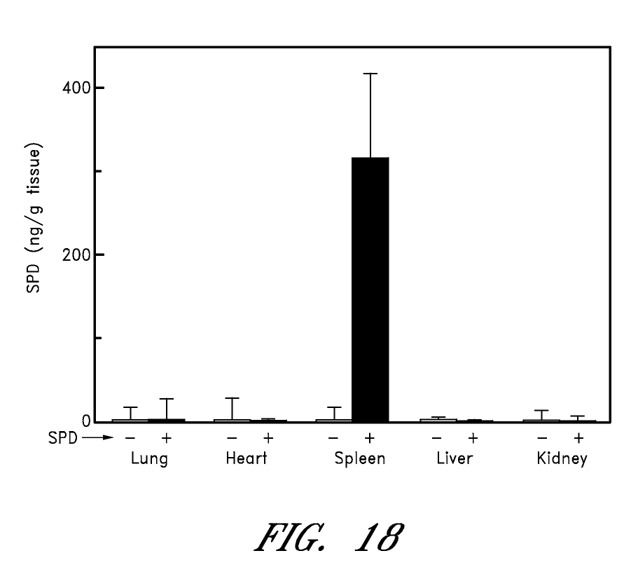

Although present at low levels in blood at baseline, multiple studies demonstrate that human plasma SP-D levels increase several fold in a variety of pro-inflammatory conditions such as pulmonary or systemic infection (Sorensen, G. L. et al., (2006) Am J Physiol Lung Cell Mol Physiol 290:L1010-L1017; Fujita, M. et al., (2005) Cytokine 31:25-33). To determine if plasma SP-D levels increase during sepsis in mice and to establish a model system for defining the origin(s) of plasma SP-D, the mouse CLP model was utilized. Sepsis was induced by a cecal ligation and puncture with a 30-gauge needle and plasma SP-D levels were measured by ELISA 48 hours after the procedure (n=5, 6-8 weeks old) (FIG. 15). Plasma SP-D levels increased several fold to a mean of approximately 40 ng/ml following CLP, indicating that systemic levels of SP-D in mice and humans respond in a similar manner. In addition, these results demonstrate that the CLP model can provide a functional in vivo system to evaluate systemic SP-D production.

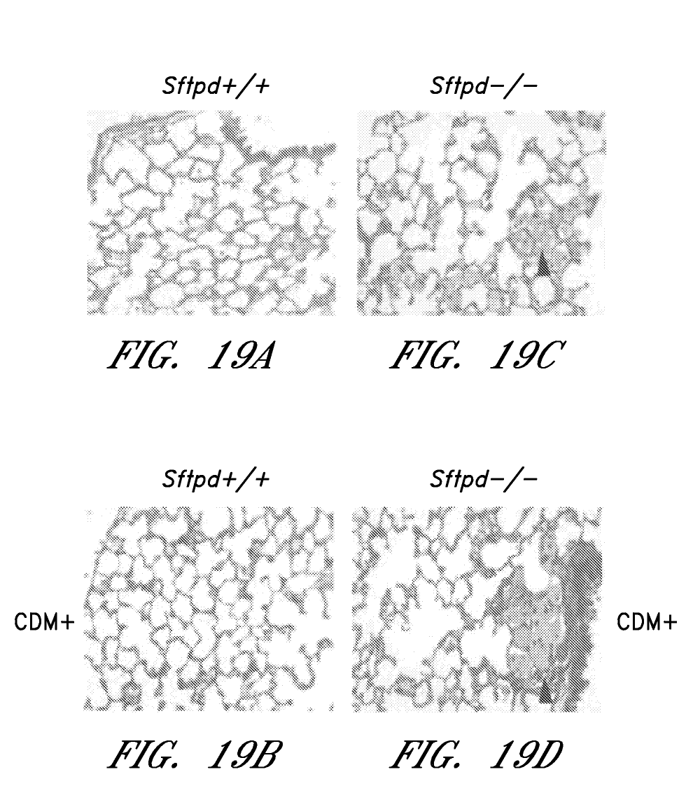

SP-D is also detected by immunostaining in vascular endothelium, stomach, small intestine, kidney, and multiple glandular tissues (Stahlman, M. T. et al., (2002) J Histochem Cytochem 50:651-660; Sorensen, G. L. et al., (2006), Am J Physiol Heart Circ Physiol 290: H2286-H2294). Although SP-D is present in several tissue types and can serve a protective role in each of these locations, SP-D circulating in plasma is the population that contributes to systemic host defense. Given the juxtaposition of the vascular endothelium to the circulating pool of SP-D and the role of vascular endothelium in host defense, the vascular endothelium can contribute to plasma SP-D pool sizes. Previous studies on Sftpd gene expression have been limited to the respiratory epithelium. Therefore, to determine if the Sftpd promoter is activated in vascular endothelial cells, a mouse fetal lung mesenchyme cell line (MFLM-91U) was utilized. These cells are derived from immortalized mouse fetal lung mesenchyme (day E19) and display characteristics of a vascular endothelial lineage (i.e. vascular endothelial growth factor receptor 2 expression and the formation of capillary-like structures with lumens when cultured on a reconstituted basement membrane) (Akeson, A. L. et al., (2000) Dev Dyn 217:11-23, which is incorporated herein by reference in its entirety). MFLM cells were transiently transfected with a plasmid that contained the Sftpd promoter coupled to a luciferase reporter gene, and Sftpd promoter activity was measured (FIG. 16). Luciferase activity increased approximately 50-fold in MFLM-91U cells transfected with the Sftpd promoter coupled to the luciferase reporter gene when compared to cells transfected with the luciferase gene alone, indicating that the Sftpd promoter is activated in vascular endothelial cells. In addition, these results support the use of this system to define the regulatory factors that keep plasma levels of SP-D several fold lower than pulmonary levels at baseline, as well as those that increase plasma SP-D levels during systemic sepsis.

In the lung, SP-D is produced by alveolar type II cells and degraded or recycled by type II cells or alveolar macrophages, resulting in a half life of 7 hours in Sftpd^{-/-} mice and 13 hours in wild type mice (Crouch, E. et al., (1992) Am J Physiol 263:L60-L66; Voorhout, W. F. et al., (1992) J Histochem Cytochem 40:1589-97; Crouch, E. et al., (1991) Am J Respir Cell Mol Biol 5:13-18; Dong, Q. and J. R. Wright, (1998) J. R. Am J Physiol 274:L97-105; Herbein, J. F. et al., (2000) Am J Physiol Lung Cell Mol Physiol 278:L830-L839; Kuan, S. F. et al., (1994) Am J Respir Cell Mol Biol 10:430-436; Ikegami, M. et al., (2000) Am J Physiol Lung Cell Mol Physiol 279:L468-L476, each of which is incorporated herein by reference in its entirety). To determine the half life of SP-D in plasma, SP-D was administered via tail vein injection and SP-D levels in plasma were measured by ELISA over time (FIG. 17). SP-D was not removed from the plasma by first pass metabolism, but rather remained in the plasma with a half life of approximately 6 hours in wild type mice. Interestingly, the plasma SP-D half life decreased to approximately 2 hours in Sftpd^{-/-} mice, whereas the half life of a truncated fragment of SP-D consisting of a trimer of only the neck and CRD has a plasma half life of 62 hours (Sorensen, G. L. et al., (2006), Am J Physiol Heart Circ Physiol 290: H2286-H2294), indicating that there is a specific cellular mechanism for uptake of plasma SP-D and that this mechanism is dependent on the N-terminus and/or collagen domain of SP-D.

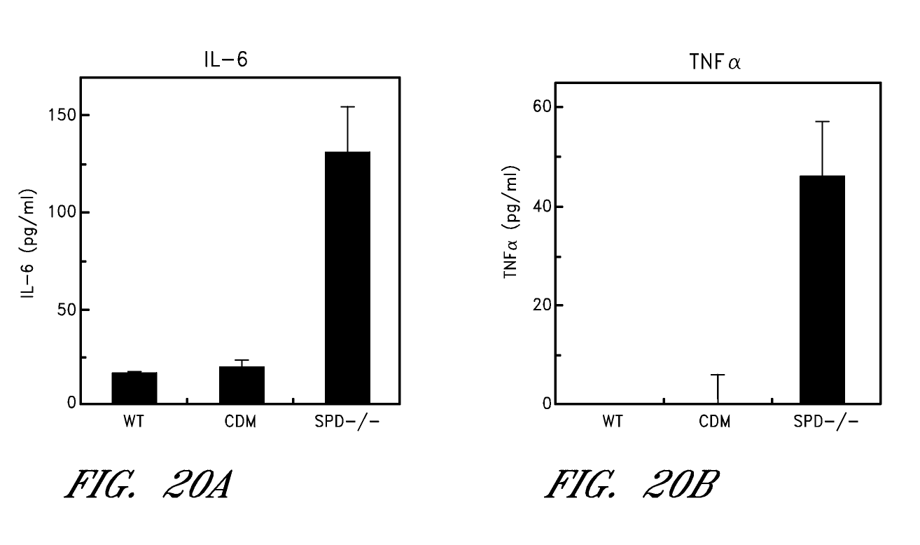

To determine the primary location of plasma SP-D uptake, SP-D was administered via tail vein injection to Sftpd^{-/-} mice, and SP-D levels in tissue homogenates were determined by SP-D ELISA 8 hours after injection (FIG. 18). Levels of SP-D in the spleen reached about 320 ng SP-D per gram of tissue, which was markedly higher than SP-D levels observed in the other tissues (and the background signal in the spleen). Therefore, although pulmonary SP-D is degraded or recycled by alveolar macrophages and type II cells, the results indicate that systemic SP-D is cleared from the circulation by the spleen.

### Mouse Studies: Role of SP-D Structural Domains in Regulating Host Defense Cells

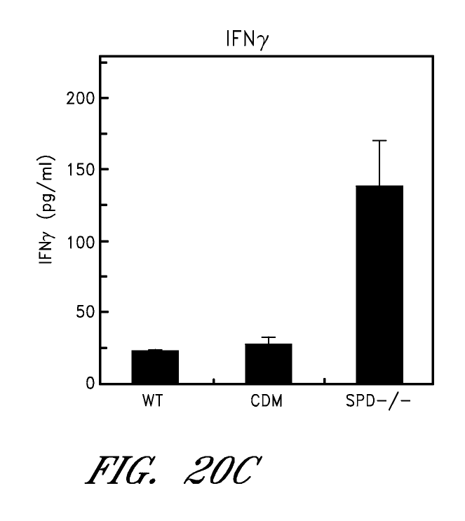

Because of the relatively large SP-D collagen domain (when compared to other collectins), SP-D collagen domain can be essential for SP-D mediated regulation of alveolar macrophages. To investigate this, an SP-D mutant protein with a normal CRD, neck domain and N-terminal domain but lacking the collagen domain (rSftpdCDM) was generated. In vitro assays demonstrated that purified rSfptdCDM formed multimers and bound carbohydrates, bacteria, and viruses in a manner that was equal to or better than the wild type protein. To determine if rSftpdCDM effectively regulated alveolar macrophage activity, the mutant transgene (rSftpdCDM^{Tg+}) was expressed in wild type and Sftpd^{-/-} mice. While the mutant protein did not disrupt pulmonary morphology or macrophage activity in wild type mice, the mutant protein failed to rescue the abnormal baseline macrophage activity characteristic of Sftpd^{-/-} mice. Enlarged foamy macrophages that expressed increased levels of metalloproteinases were readily observed in Sftpd^{-/-} mice and Sftpd^{-/-} mice that expressed the rSftpdCDM protein (rSftpdCDM^{Tg+}/Sftpd^{-/-}) (FIG. 19).

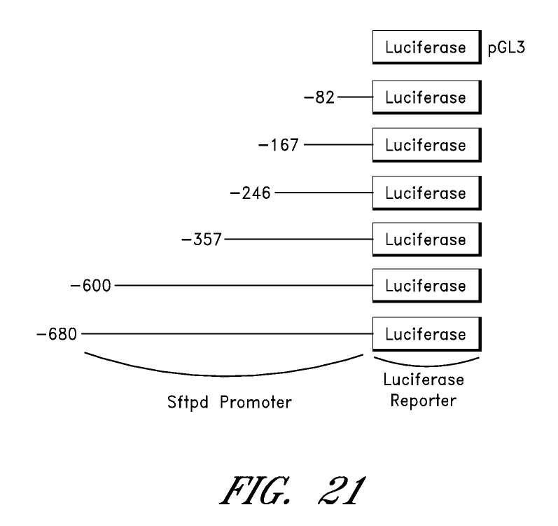

To determine if rSftpdCDM regulates alveolar macrophage activity during infectious challenge, the response of wild type, Sftpd^{-/-}, and rSftpdCDM^{Tg+}/Sftpd^{-/-} mice to intratracheal exposure to influenza A virus (IAV) was evaluated. In contrast to Sftpd^{-/-} mice, no detectable IAV was recovered from the wild type or rSftpdCDM^{Tg+}/Sftpd^{-/-} lung homogenates. In addition, the increased IL-6, TNFα, and IFN-γ levels observed in IAV challenged Sftpd^{-/-} mice were restored to wild type levels in rSftpdCDM^{Tg+}/Sftpd^{-/-} mice (FIG. 20). Taken together, these results indicate that although rSftpdCDM does not effectively regulate baseline alveolar macrophage activity, rSftpdCDM can facilitate a normal alveolar macrophage response during viral challenge. Moreover, the rSftpdCDM mutant protein provides a model system to determine if the SP-D structural domains that elicit the systemic anti-inflammatory properties of SP-D in LPS-induced inflammation parallel those required during infectious challenge in the lung.

The binding of SP-D to E. coli LPS has been demonstrated both in vivo and in vitro (Kuan et al., (1992) J Clin Invest, 90:97-106; Lim et al., (1994) Biochem Biophys Res Commun, 202:1674-1680; van Rozendaal et al., (1999) Biochim Biophys Acta, 1454:261-269; Crouch et al., (1998) Am J Respir Cell Mol Biol, 19:177-201; Pikaar et al., (1995) J Infect Dis, 172:481-489 each of which is incorporated herein by reference in its entirety). Premature newborns are deficient in surfactant, including SP-D (Miyamura et al., (1994) Biochim Biophys Acta, 1210:303-307, which is incorporated herein by reference in its entirety). The commercially available surfactants for treatment of the newborn with respiratory distress syndrome contain SP-B and SP-C, but do not contain SP-A or SP-D. Increased inflammatory responses seen in the premature newborn lung can result from a deficiency in host defenses, including low levels of SP-A and SP-D and a relatively low number of macrophages (Awasthi et al., (1999) Am J Respir Crit Care Med, 160:942-949, which is incorporated herein by reference in its entirety). Fetal inflammation associated with chorioamnionitis and postnatal infection of the lung are associated with the development of chronic lung injury and bronchopulmonary dysplasia (Li et al., (2002) Microbes Infect, 4:723-732, which is incorporated herein by reference in its entirety).

The finding that SP-D ameliorated systemic effects and prevented death following intratracheally administered LPS supports the concept that SP-D binds to LPS and detoxifies or inhibits LPS transit from the pulmonary to the systemic compartment. Similar to findings in premature human newborns, septic shock is also a relatively frequent cause of mortality in adults (Manocha et al., (2002) Expert Opin Investig Drugs, 11:1795-1812, which is incorporated herein by reference in its entirety). As in the premature lung, increased permeability occurs following injury and ventilation of the adult lung (Sartori et al., (2002) Eur Respir J, 20:1299-1313; Lecuona et al., (1999) Chest, 116:29S-30S, each of which is incorporated herein by reference in its entirety). Thus, SP-D represents a potential therapeutic strategy for prevention of the systemic inflammatory response originating from a lung with infection.

As shown herein, rhSP-D can be safely administered intratracheally to prevent pathogen-induced systemic endotoxin shock in the premature newborn lamb. Such a therapy can be useful in protecting newborns from pulmonary infection and its sequelae.

In addition, the studies described herein demonstrate that: 1) SP-D scavenges LPS from the systemic circulation and inhibits LPS induced systemic inflammation, 2) SP-D inhibits LPS-induced inflammation by direct SP-D/LPS interactions, 3) systemic LPS-induced inflammation is reduced in Sftpd^{-/-} mice, 4) SP-D reduces inflammation and improves survival in mice during live systemic bacterial challenge, 5) plasma SP-D levels increase during sepsis in mice, 6) vascular endothelial cells express the Sftpd gene, 7) systemic SP-D is cleared by the spleen, and 8) unique SP-D structural domains regulate alveolar macrophages. Furthermore, as shown herein, experimental models of intravenous LPS injection, CLP, and vascular endothelial Sftpd expression are established and functional in the laboratory.

Accordingly, an SP-D polypeptide or biologically active fragment thereof, or a nucleic acid encoding the same, can be administered to an individual to prevent or treat pulmonary infections and/or sepsis. In some embodiments, SP-D treatment can, for example, inhibit LPS-induced inflammation such that it improves survival or tissue injury derived from administration or introduction of lethal doses of LPS into a mammal. In other embodiments, SP-D treatment can, for example, inhibit LPS-induced inflammation by enhancing clearance of LPS from plasma. In still other embodiments, SP-D treatment can, for example, prevent leakage of LPS from the respiratory tree into the systemic circulation in the absence of lung injury when administered to the lungs. Embodiments of SP-D treatment can also be used, for example, for the treatment of sepsis by administering an SP-D polypeptide or a biologically active fragment thereof, or a nucleic acid encoding the same, in a systemic manner to prevent or treat polymicrobial sepsis or bacterial challenge. In still other embodiments, SP-D treatment can, for example, be administered to the lungs or in a systemic manner to treat acute respiratory distress syndrome.

SP-D treatment can be used alone or in conjunction with other treatments, such as antibiotic administration. Further, in some embodiments, nucleic acids encoding SP-D or fragments thereof can be administered to an individual. The nucleic acid encoding SP-D can be, for example, contained within an adenoviral vector. The adenoviral vector can be constructed, for example, according to the methods described in PCT Application No. PCT/US02/35121, which is incorporated herein by reference in its entirety.

The SP-D protein can be, for example, recombinant SP-D. In some embodiments, the recombinant SP-D is a recombinant human SP-D (rhSP-D). For example, in some embodiments, the SP-D polypeptide is the mature polypeptide sequence of Accession No. NP_{—}003010 (SEQ ID NO: 2). In further embodiments, the SP-D protein can be, for example, the SP-D precursor sequence of Accession No. NP_{—}003010 (SEQ ID NO: 3). In some embodiments, the SP-D protein can be prepared from, for example, the nucleic acid encoding SP-D or a fragment thereof that can be transfected to any suitable organism in order to prepare SP-D protein or fragments thereof in bulk. The protein can then be isolated and purified using methods known in the art. The term “purified” does not require absolute purity; rather, it is intended as a relative definition. Isolated proteins have been conventionally purified to electrophoretic homogeneity by Coomassie staining, for example. Purification of starting material or natural material to at least one order of magnitude, preferably two or three orders, and more preferably four or five orders of magnitude is expressly contemplated.

The term “polypeptide” can refer, for example, to a polymer of amino acids without regard to the length of the polymer; thus, peptides, oligopeptides, and proteins are included within the definition of polypeptide. This term also does not specify or exclude prost-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

In some embodiments of the invention, the term “purified” describes an SP-D polypeptide of the invention which has been separated from other compounds including, but not limited to nucleic acids, lipids, carbohydrates and other proteins. A polypeptide is substantially pure when at least about 50%, preferably 60 to 75% of a sample exhibits a single polypeptide sequence. A substantially pure polypeptide typically comprises about 50%, preferably 60 to 90% weight/weight of a protein sample, more usually about 95%, and preferably is over about 99% pure. Polypeptide purity or homogeneity is indicated by a number of means well known in the art, such as agarose or polyacrylamide gel electrophoresis of a sample, followed by visualizing a single polypeptide band upon staining the gel. For certain purposes higher resolution can be provided by using HPLC or other means well known in the art.

In some embodiments of the present invention, the SP-D sequence can be derived from the nucleic acid precursor sequence Accession No. NM_{—}003019 (SEQ ID NO: 1).

The term “substantially homologous”, when used herein with respect to an SP-D encoding nucleotide sequence, refers to a nucleotide sequence corresponding to a reference nucleotide sequence, wherein the corresponding sequence encodes a polypeptide having substantially the same structure as the polypeptide encoded by the reference nucleotide sequence. In some embodiments, the substantially similar nucleotide sequence encodes the polypeptide encoded by the reference nucleotide sequence.

In the context of the present invention, “substantially homologous” can refer to nucleotide sequences having at least 50% sequence identity, or at least 60%, at least 70%, at least 80%, at least 85%, at least 90%, at least 95%, at least 96%, at least 97%, or at least 99% sequence identity compared to a reference sequence that encodes a protein having at least 50% identity, or at least 85%, at least 90%, at least 95%, at least 96%, at least 97%, or at least 99% sequence identity to a region of sequence of a reference protein. Also, “substantially homologous” preferably also refers to nucleotide sequences having at least 50% identity, more preferably at least 80% identity, still more preferably 95% identity, yet still more preferably at least 99% identity, to a region of nucleotide sequence encoding a reference protein. The term “substantially homologous” is specifically intended to include nucleotide sequences wherein the sequence has been modified to optimize expression in particular cells.

A polynucleotide comprising a nucleotide sequence “substantially homologous” to the SP-D nucleotide sequence preferably hybridizes to a polynucleotide comprising the reference nucleotide sequence. The reference nucleotide sequence can be, for example, the nucleic acid precursor sequence Accession No. NM_{—}003019 (SEQ ID NO: 1) or a fragment thereof. The term “hybridize” refers to a method of interacting a nucleic acid sequence with a DNA or RNA molecule in solution or on a solid support, such as cellulose or nitrocellulose. If a nucleic acid sequence binds to the DNA or RNA molecule with high affinity, it is said to “hybridize” to the DNA or RNA molecule.

A pharmaceutical preparation comprising SP-D protein or fragments thereof, or nucleic acids encoding them, can be prepared following methods known in the art. In some embodiments of the present invention, the SP-D protein or nucleic acid or the fragment or analog or derivative thereof can be introduced into the subject in the aerosol form in an amount between about 0.01 mg per kg body weight of the mammal up to about 100 mg per kg body weight of said mammal. In some embodiments, the dosage can be, for example, from about 0.05, 0.1, 0.5 to about 25, 50, 75, or 100 mg/kg. In farther embodiments, the dosage can be in a range of from about 0.75, 1.0, 1.5, or 2.0 to about 5.0, 7.5, 10, or 20 mg/kg. In a specific embodiment, the dosage is dosage per day. One of ordinary skill in the art can readily determine a volume or weight of aerosol corresponding to this dosage based on the concentration of SP-D protein or nucleic acid in an aerosol formulation of the subject matter. Alternatively, one can prepare an aerosol formulation with the appropriate dosage of SP-D protein or nucleic acid in the volume to be administered, as is readily appreciated by one of ordinary skill in the art. In some embodiments of the present invention, administration of SP-D protein or nucleic acid directly to the lung allows use of less SP-D protein or nucleic acid, thus limiting both cost and unwanted side effects.

In some embodiments of the present invention, a pharmaceutical preparation comprising the SP-D protein or nucleic acid or the fragment or analog or derivative thereof can be introduced into the subject in a systemic manner in an amount between about 0.01 mg per kg body weight of the mammal up to about 100 mg per kg body weight of said subject. In some embodiments, the dosage can be, for example, from about 0.05, 0.1, 0.5 to about 25, 50, 75, or 100 mg/kg. In further embodiments, the dosage can be in a range of from about 0.75, 1.0, 1.5, or 2.0 to about 5.0, 7.5, 10, or 20 mg/kg. In a specific embodiment, the dosage is dosage per day. One of ordinary skill in the art can readily determine a volume or weight of a pharmaceutical preparation corresponding to this dosage based on the concentration of SP-D protein or nucleic acid in said pharmaceutical preparation of the subject matter. Alternatively, one can prepare a pharmaceutical formulation with the appropriate dosage of SP-D protein or nucleic acid in the volume to be administered, as is readily appreciated by one of ordinary skill in the art.

The SP-D of the present invention, combined with a dispersing agent, or dispersant, can be administered in an aerosol formulation as a dry powder or in a solution or suspension with a diluent. In some embodiments of the present invention, formulations comprising SP-D protein or nucleic acid can be prepared for use in a wide variety of devices that are designed for the delivery of pharmaceutical compositions and therapeutic formulations to the respiratory tract. In some embodiments, the preferred route of administration is in the aerosol or inhaled form. The SP-D of the present invention can also, for example, be administered systemically in a solution or suspension with a diluent. In some embodiments of the present invention, formulations comprising SP-D protein or nucleic acid can be prepared for use in a wide variety of devices that are designed for the systemic delivery of pharmaceutical compositions and therapeutic formulations. In some embodiments, the preferred route of administration is by systemic delivery. The formulation can be administered in a single dose or in multiple doses depending on the disease indication. It will be appreciated by one of skill in the art that the exact amount of prophylactic or therapeutic formulation to be used will depend on the stage and severity of the disease, the physical condition of the subject, and a number of other factors.

In some embodiments, the SP-D formulation can also contain other agents to treat sepsis or a pulmonary infection, such as, for example, oral or intravenously administered antibiotics.

### EXAMPLES

The following examples are offered to illustrate, but not to limit, the claimed invention.

### Example 1

### Preparation and Purification of Recombinant SP-D

rhSP-D was synthesized by transfection of CHO DHFR cells with a cDNA encoding full-length human SP-D. Transfected cells were selected with increasing concentrations of methotrexate. Transfected pools were cloned by limiting dilution and high expressing clones were identified using an ELISA designed specifically for this purpose. An SP-D clone was grown in roller bottles in medium containing serum and then switched to JRH EX-CELL 302 medium for bioproduction. The choice of the serum-free medium was found to be key in achieving high production levels of rhSP-D. To avoid high shear rates associated with large-scale buffer exchange methods, the protein was captured from conditioned medium using anion ion exchange chromatography to concentrate the sample and remove glucose. Specifically, the medium was diluted, pH adjusted to 7.4, and then loaded on a Q ceramic hyperD F resin (Ciphergen, Fremont, Calif.). Following extensive washing to remove impurities, the rhSP-D was eluted using 25 mM Tris, 1.2 M NaCl, pH 7.4. Eluted material was diluted and calcium was added to a final concentration of 5 mM. The rhSP-D was then affinity purified on maltose agarose using previously described methods (Hartshorn et al., (1996) Am J Physiol Lung Cell Mol Physiol, 271:L753-L762, which is incorporated herein by reference in its entirety). To minimize endotoxin levels in the final preparation, the anion exchange resin and all chromatography equipment was sanitized by exposure to 0.2 N NAOH and the maltose agarose was treated with an acid-ethanol mixture. Purified rhSP-D migrated as a multimer of greater than 1×10⁶ daltons on size exclusion chromatography. On SDS-PAGE gels, the protein migrated as a trimer under nonreducing conditions and fully converted to an ˜48 kDa monomeric form when reduced. Recombinant hSP-D bound and aggregated E. coli in vitro in a calcium-dependent manner (data not shown). The rhSP-D used in these experiments was at a concentration of 0.5 mg/ml in 20 mM Tris, 200 mM NaCl, 1 mM EDTA pH 7.4. The endotoxin level in the rhSP-D preparations ranged from 0.1-0.5 EU/ml (Limulus Lysate Assay, Charles River Laboratories, Wilmington, Mass.). In a preliminary study, instilling a treatment dose of rhSP-D into normal adult mice and premature lambs did not induce lung inflammation (data not shown). Thus, the endotoxin level in rhSP-D either was below levels that induce inflammation or the endotoxin present was bound to rhSP-D and unable to elicit a response.

### Example 2

### Purification of Endogenous SP-D

Endogenous SP-D is purified from bronchoalveolar lavage fluid as previously described (Kingma, P. S. et al., (2006) J Biol Chem 281:24496-24505; Strong, P. et al., (1998) J Immunol Methods 220:139-149, each of which is incorporated herein by reference in its entirety). Lavage fluid is cleared of lipid by centrifugation. The lipid-free supernatant is applied to a 20 ml maltosyl-Sepharose column in 20 mM Tris-HCl (pH 7.4), 5 mM CaCl₂. The column is washed with a solution of 20 mM Tris-HCl (pH 7.4), 5 mM CaCl₂, and 1 M NaCl, followed by a selective elution of SP-D with manganese chloride. The pooled fractions are diluted 10-fold in a solution of 20 mM Tris-HCl (pH 7.4) and 30 mM CaCl₂ and applied to a 1 ml bed volume maltosyl-Sepharose column. The column is stripped of LPS with a solution of 20 mM Tris-HCl (pH 7.4), 20 mM n-octyl-d-glucopyranoside, 200 mM NaCl, 2 mM CaCl₂ and 100 μg/ml polymyxin and washed with a solution of 20 mM Tris-HCl (pH 7.4), 0.5 mM CaCl₂ and 200 mM NaCl. SP-D is eluted with a solution of 20 mM Tris-HCl (pH 7.4), 200 mM NaCl, and 1 mM EDTA. Under the conditions described, LPS concentration is typically ≦0.1 endotoxin units/μg protein.

### Example 3

### Preparation of Premature Lambs for Treatment

All animals were delivered by Cesarean section at 130 d gestation age from Suffolk ewes bred to Dorset rams (term 150 d GA) as previously described (Kramer et al., (2002) Am J Respir Crit Care Med, 165:463-469; Kramer et al., (2001) Am J Respir Crit Care Med, 163:158-165, each of which is incorporated herein by reference in its entirety). After exposure of the fetal head and neck, an endotracheal tube was tied into the trachea. The fetal lung fluid that could be easily aspirated by syringe was recovered and the lambs were delivered and weighed.

### Example 4

### LPS Exposure to the Ventilated Premature Lamb

Before the first breath the lambs received 0.1 mg/kg E. coli LPS (E. coli 055:B5, Sigma, St. Louis, Mo.) mixed with 1 ml (25 mg) Survanta (Ross Products Division, Abbott Laboratories, Columbus, Ohio), followed by 10 ml air given into the airways by syringe. LPS was mixed with small amounts of surfactant and given before the first breath lung to facilitate uniform distribution of LPS in the lung. During and after the first breath LPS is then distributed to the peripheral airways. Ten ml of air was administered via the trachea after LPS instillation to enhance the clearance of fetal lung fluid and to prevent mixing of LPS with rhSP-D prior to distribution of LPS to the peripheral airways. Twenty-five mg Survanta was used to instill the endotoxin.

### Example 5

### Administration of RHSP-D to the LPS-Exposed Premature Lamb Lungs

LPS-exposed lambs as described above were then treated a dose of Survanta either combined with rhSP-D (treatment group) or without rhSP-D (control group). The treatment dose of Survanta was adjusted to provide a total of 100 mg/kg. This later dose of Survanta was instilled via the tracheal tube with either 12 ml of buffer containing 2 mg/kg rhSP-D (treatment group) or with 12 ml buffer only (control group). All animals were ventilated for 5 h with time-cycled and pressure-limited infant ventilators (Sechrist Industries, Anaheim, Calif.) using similar ventilation strategies. A 5 F catheter was advanced into the aorta via an umbilical artery and a 10 ml/kg transfusion of filtered fetal blood collected from the placenta was administered within 10 min of delivery to correct low hematocrit associated with prematurity. Blood pressure, heart rate, tidal volume (VT) (CP-100: Bicore Monitoring Systems, Anaheim, Calif.) and body temperature were monitored continuously. Blood gas, pH, base excess (BE), hematocrit, potassium, calcium and glucose levels were analyzed by a blood gas, electrolyte and metabolite system (Radiometer Copenhagen USA, West Lake, Ohio) at least every 20 min or when ventilatory status changed as indicated by changes in chest movement and tidal volumes. Rate of 40 breaths/min: inspiratory time: 0.6 s, positive end expiratory pressure (PEEP)=4 cmH20 were not changed. Peak inspiratory pressure (PIP) was changed to maintain VT at 8-9 ml/kg. Pressure was limited to PIP 35 cmH20 to avoid pneumothorax. Fraction of inspired oxygen (Fio2) was adjusted to keep a target pO2 of 100-150 mmHg. Ten percent dextrose (100 ml/kg/d) was infused continuously through the arterial catheter. Dynamic compliances were calculated from VT measured with a pneumotachometer that was normalized to body weight and divided by the ventilatory pressure (PIP-PEEP). Rectal temperature was maintained at the normal body temperature for sheep (38.5° C.) with heating pads, radiant heat and plastic body covering wrap. Supplemental ketamine (10 mg/kg intramuscularly) and acepromzaine (0.1 mg/kg intramuscularly) was used to suppress spontaneous breathing.

### Example 6

### Preparation for Lung Processing

After five hours, the lambs were deeply anesthetized with 25 mg/kg pentobarbital intravenously and ventilated briefly with 100% oxygen. The endotracheal tube was clamped for 3 min to permit oxygen absorption to render the lung airless. For the lambs that did not survive the 5 h study period, death was determined by either systolic blood pressure of lower than 10 mm/Hg or the absence of heart a beat.

### Example 7

### Data Analysis

Results are given as means±SEM. rhSP-D treatment groups and buffer control groups were compared using two-tailed t tests. Log-rank tests were used for percentage of survival comparison between groups. Significance was accepted at p<0.05.

### Example 8

### Processing of Lungs

The thorax was opened, the lungs were inflated with air to 40 cm H₂0 pressure for 1 min, and the maximal lung volume recorded. The lungs were deflated and lung gas volume was measured at 20, 15, 10, 5 and 0 cm H₂0. Lung tissue of the right lower lobe was frozen in liquid nitrogen for RNA isolation. Bronchoalveolar lavage (BAL) was performed on the left lung by filling it with 0.9% NaCl at 4° C. until visually distended, and the lavage was repeated five times. BAL fluid (BALF) was pooled and aliquots saved for determination of total protein (Lowry et al. (1951), J Biol Chem 1951;193:265-275, which is incorporated herein by reference in its entirety).

### Example 9

### Preparation of Alveolar Cells

BALF was centrifuged at 500×g for 10 min and the cells in the pellets were counted using trypan blue. Differential cell counts were performed on stained cytospin preparations (Diff-Quick; Scientific Products, McGraw Park, Ind.). Activation of the cells recruited to the airways was assessed by measuring hydrogen peroxide using an assay based on the oxidation of ferrous iron (Fe²⁺) to ferric iron (Fe³⁺) by hydrogen peroxide under acidic conditions (Bioxytech H₂O₂-560 assay; OXIS International, Portland, Oreg.).

Apoptotic cells were detected by annexin V and proprium iodide staining (Pharmigen, Mountain View, Calif.) and analyzed by flow cytometry as described previously (Kramer et al. (2001), Am J Physiol Lung Cell Mol Physiol, 280:L689-L694, which is incorporated herein by reference in its entirety).

### Example 10

### Measurement of RHSP-D in BALF, Lung Tissue and Serum

Levels of rhSP-D in BALF, the supernatant of lung homogenate after centrifugation and in serum collected at 5 h of age were analyzed by ELISA. For immunoblotting, 10 μl of BALF was loaded on a SDS/PAGE gel, transferred to nitrocellulose and the blots probed with rabbit anti-rhSP-D serum that does not crossreact with ovine SP-D, allowing an estimate of the level of exogenous rhSP-D in the samples.

### Example 11

### Lung Histology Methods

The right upper lobe was inflation fixed with 10% formalin at 30 cm H₂0 pressure. Paraffin embedded tissues were sectioned (9 μm) and stained with hematoxylin and eosin. Immunohistochemical detection of IL-6, IL-8 and IL-1β on lung tissues was performed as previously described (Ikegami et al., (2004) Am J Physiol Lung Cell Mol Physiol, 286:L573-L579, which is incorporated herein by reference in its entirety) using rabbit polyclonal antibody for ovine IL-6 (Chemicon, Temecula, Calif.), mouse polyclonal antibody for ovine IL-8 (Chemicon) and rabbit polyclonal antibody for ovine IL-1β.

### Example 12

### Measurement of Endotoxin and Cytokine Levels in Plasma

LPS was quantified in plasma at 0 (cord blood), 30 min, 1 h, 2 h and 5 h with the Limulus amebocyte lysate assay (Bio Whittaker, Walkersville, Md.). ELISA was used to determine IL-8 and IL-1β in plasma using antibodies from Chemicon.

### Example 13

### RNA Analysis in Lung, Spleen and Liver

Total RNA was isolated from the right lower lung lobe, spleen and the liver by guanidinium thiocyanate-phenol-chloroform extraction. Spleen and liver tissue were used to evaluate whether the intratracheally-administered LPS induced a systemic inflammatory response. RNase protection assays were performed using RNA transcripts of ovine IL-6, IL-1β, IL-8, IL-10 and TNFα as described previously (Naik et al., (2001) Am J Respir Crit Care Med 2001; 164:494-498, which is incorporated herein by reference in its entirety). Ovine ribosomal protein L32 was the reference RNA. Densities of the protected bands were qualified on a phosphorimager using ImageQuant software (Molecular Dynamics Inc., Sunnyvale, Calif.).

### Example 14

### Prevention of Sepsis in Newborns by Administration of RHSP-D

A newborn human at risk for sepsis is identified. The newborn is administered rhSP-D using an aerosol formulation at lmg SP-D per kg body weight. The administration is performed 4 times per day. The patient is monitored continuously. By use of this method, the susceptibility of the newborn to sepsis is decreased.

### Example 15

### Treatment of Sepsis in an Infant by Administration of RHSP-D

An infant diagnosed with sepsis is identified. The infant is administered rhSP-D at 4 mg rhSP-D per kg body weight using an aerosol formulation. The administration is performed every other hour. Plasma endotoxin levels are monitored. By use of this method, the sepsis subsides and the risk of death is decreased.

### Example 16

### Treatment of Sepsis in an Infant by Administartion of 30 AA Fragment of RHSP-D

An infant diagnosed with sepsis is identified. The infant is administered a 30 amino acid peptide corresponding to a region of SP-D at 0.5 mg peptide per kg body weight using an aerosol formulation. The administration is performed every hour. The patient health is monitored continuously. By use of this method, the sepsis subsides and the risk of death is decreased.

### Example 17

### Treatment of a Lung Infection to Prevent Risk of Death of Sepsis in an Individual by Administration of RHSP-D

An individual with a severe lung infection is identified. The individual is at risk of developing sepsis if the lung infection continues. The patient is administered rhSP-D at 10 mg/kg, administered two times per day. Endotoxin levels in patient plasma are measured twice a day for 5 days. Patient health is monitored continuously. By use of this method, the lung infection subsides, and the risk of developing sepsis decreases.

### Example 18

### Treatment of a Lung Infection to Prevent Risk of Death of Sepsis in an Individual by Administration of RHSP-D in Combination with an Antibiotic

An individual with a severe lung infection is identified. The individual is at risk of developing sepsis if the lung infection continues. The patient is administered rhSP-D at 1 mg/kg, administered 6 times per day. The patient is also given an oral antibiotic treatment. Endotoxin levels in patient plasma are measured twice a day for 5 days. Patient health is monitored continuously. By use of this method, the lung infection subsides, and the risk of developing sepsis decreases.

### Example 19

### Protocol for LPS and SP-D Infection Studies

Mice are warmed and anesthetized with inhaled 2% isoflurane. Anesthesia is confirmed by the toe pinch test. Tails are prepared with alcohol and injected with control buffer, SP-D, LPS, or LPS with SP-D that are pre-incubated at room temperature for 10 minutes. SP-D (1 mg/ml) is stored in SP-D buffer (20 mM Tris-HCl (pH 7.4), 200 mM NaCl, 1 mM EDTA) and is diluted in PBS with 1 mM CaCl₂. LPS is stored in an equal volume of SP-D buffer and is diluted in PBS with 1 mM CaCl₂. PBS with 1 mM CaCl₂ and an equal volume of SP-D buffer is used as control buffer.

### Example 20

### Preparation of Plasma and Organs for SP-D Analysis

After administration of LPS, SP-D or control buffer, mice are given a lethal dose of thiopentone sodium (80 μg/g), and blood is collected by cardiac puncture or by retro-orbital technique. The blood is placed on ice and spun immediately to isolate plasma. The heart, lung, liver, spleen, and kidneys are harvested and placed in paraformaldehyde for histology or homogenized for RNA isolation.

### Example 21

### Systemic SP-D Treatment Improves Survival in an LPS-Infected Mammal

Mice are given a lethal dose of LPS (8 mg/kg) with SP-D (2 mg/kg) or control buffer via tail vein injection as described in Example 19. Survival is monitored every 4 hours for 72 hours. Animals in a moribund state (ruffled fur, complete inability to move, and diarrhea) are considered nonsurvivors and euthanized with a lethal dose of thiopentane sodium. Studies predict a 75% mortality rate by 72 hours in LPS treated mice. By use of this method, a statistically significant difference in survival at 72 hours between treatment groups is observed, with higher survival rates observed in the SP-D-treated group, indicating that systemic SP-D treatment improves survival of an LPS-infected mammal.

### Example 22

### Systemic SP-D Treatment Improves Tissue Injury in an LPS-Infected Mammal

Mice are treated with LPS (4 mg/kg) with SP-D (2 mg/kg) or control buffer via tail vein injection as described in Example 19. Livers are harvested at 24 hours and markers of tissue injury, including but not limited to hepatic TNFα, NFκB, iNOS and myeloperoxidase expression, hepatocellular necrosis and neutrophil infiltration, are evaluated. For gene expression studies, livers are homogenized, and RNA is isolated and tested for concentration and purity. cDNA is synthesized by reverse transcriptase polymerization and amplified by PCR. Gene expression is quantified by real time PCR or densitometry of the PCR product following resolution on agarose gels. All results are reported relative to L32 or GAPDH controls. By use of this method, a statistically significant decrease in the markers of LPS-induced tissue injury is observed in SP-D treated mice, indicating that systemic SP-D treatment improves tissue injury in an LPS-infected mammal.

### Example 23

### SP-D Treatment Increases Clearance Rates of Plasma LPS

Mice are treated with LPS (5 μg/kg) with control buffer or SP-D (150 μg/kg) as described in Example 19. Blood is collected at 0.5, 1, 2, 4, and 6 hours after injection. LPS levels are monitored by limulus assay as described in Example 12, and the LPS half-life is calculated. By use of this method, a statistically significant increase in clearance rates and a statistically significant decrease in LPS half-life is observed in SP-D treated mice, indicating that SP-D treatment increases clearance rates of plasma LPS.

### Example 24

### SP-D Treatment Inhibits LPS-Induced Inflammation in Tissue-Specific Locations

Mice are treated with LPS (5 μg/kg) with control buffer or SP-D (150 μg/kg) as described in Example 19. Organs, including but not limited to the heart, lung, liver, spleen, and kidney, are harvested 2 hours after injection, and mRNA is isolated from tissue homogenates. IL-6 gene expression is measured by real time PCR. By use of this method, a statistically significant decrease in LPS-stimulated IL-6 expression is observed in specific tissues of SP-D treated mice, indicating that SP-D treatment inhibits LPS-induced inflammation in tissue-specific locations.

### Example 25

### SP-D Treatment Inhibits LPS-Induced Inflammation in Specific Cell Types

Single cell suspensions of splenic leukoctyes are isolated from mouse spleens by separation in a 100-μm strainer and placed in tissue culture media. Optionally, further selection of splenic leukocytes into lymphocyte and macrophage populations is accomplished by adherence to tissue culture plates. Following culture in LPS-free conditions for 48 hours, leukocytes are stimulated with LPS (1 μg/ml) or LPS with SP-D (5 μg/ml) for 24 hours. Media is collected and IL-6 and TNFα levels are measured by ELISA in culture supernatants. By use of this method, a statistically significant decrease in IL-6 and TNFα levels is observed in splenic leukocytes treated with SP-D, indicating that SP-D treatment inhibits LPS-induced inflammation in specific cell types.

### Example 26

### SP-D Treatment Prevents Systemic Leak of LPS in the Absence of Lung Injury

Wild type and Sftpd^{-/-} mice are anesthetized with inhaled isoflurane, and LPS (1 mg/kg) is administered by intratracheal injection. Blood is harvested at 1, 2, 4, and 6 hours after injection, and plasma LPS levels are measured by limulus assay. By use of this method, a statistically significant difference in plasma LPS levels is observed between the two groups, with higher LPS levels observed in Sftpd^{-/-} mice, indicating that SP-D treatment can prevent systemic leak of LPS in the absence of lung injury.

### Example 27

### Protocol for Cecal Ligation and Puncture (CLP)

Mice are anesthetized with inhaled 2% isoflurane or by non-lethal intraperitoneal injection of thiopentone sodium. After sterile preparation, the mouse cecum is exteriorized via a 2-cm abdominal incision and ligated approximately 0.5 cm distal to the ileocecal valve. The ligated cecum is punctured with a 25- or 30-gauge needle. The cecum is replaced in the abdomen, and the abdomen is closed. One ml of normal saline solution is injected subcutaneously to compensate for third-space fluid losses. Sham mice are treated as described above except that the cecum is isolated and returned to the abdomen without ligation or puncture. Immediately following CLP, mice are prepared for injection as described in Example 19.

### Example 28

### SP-D Treatment Improves Survival in Systemic Infections

CLP is performed on mice as described in Example 27. Subsequently, the mice are administered SP-D (2 mg/kg) or control buffer via tail injection as described in Example 19. Survival is monitored every 4 hours for 72 hours. Animals in a moribund state (ruffled fur, complete inability to move, and diarrhea) are considered non-survivors and are euthanized with a lethal dose of thiopentane sodium. By use of this method, a statistically significant difference in survival at 72 hours between treatment groups is observed, with higher survival rates observed in the SP-D-treated group, indicating that SP-D treatment improves survival in a systemically infected subject.

### Example 29

### SP-D Treatment Reduces Tissue Injury During Systemic Infections

CLP is performed on mice with and without SP-D as described in Example 27 and Example 28. The liver is harvested at 24 hours, and markers of tissue injury are evaluated as described in Example 22. By use of this method, a statistically significant decrease in the markers of LPS-induced tissue injury is observed in SP-D treated mice, indicating that SP-D treatment reduces tissue injury in a systemically infected subject.

### Example 30

### SP-D Treatment Enhances the Immune Response in Systemic Infections

CLP is induced in C57BL/6 mice with and without SP-D as described in Example 27 and Example 28. The peritoneal cavity is lavaged, and blood is collected 6 hours after CLP. Plasma and peritoneal wash LPS levels are determined by limulus assay. Bacteria counts are determined by serial log dilutions of the blood or peritoneal wash and plating on tryptic soy agar dishes. Colonies are counted after overnight incubation. By use of this method, a statistically significant difference in plasma and peritoneal LPS or bacterial levels is observed between the two groups, with lower LPS or bacterial levels observed in SP-D treated mice, indicating that SP-D treatment enhances the immune response in a systemically infected subject.

### Example 31

### SP-D Treatment Decreases the Systemic Spread of LPS or Bacteria

CLP is induced in C57BL/6 mice with and without SP-D as described in Example 27 and Example 28. The peritoneal cavity is lavaged, and blood is collected 6 hours after CLP. Plasma and peritoneal wash LPS levels are determined by limulus assay. Bacteria counts are determined by serial log dilutions of the blood or peritoneal wash and plating on tryptic soy agar dishes. Colonies are counted after overnight incubation. By use of this method, a statistically significant difference in LPS or bacterial levels in only plasma is observed between the two groups, with lower LPS or bacterial levels observed in SP-D treated mice, indicating that SP-D treatment decreases the systemic spread of LPS or bacteria.

### Example 32

### Markers for Acute Respiratory Distress Syndrome (ARDS) are Increased in SFTPD^{-/-} Mice Suffering from Sepsis

Wild type and Sftpd^{-/-} mice are subjected to CLP as described in Example 27. Markers of ARDS (for example, including but not limited to alveolar protein levels, Sat PC levels, or neutrophil infiltrate) are measured as follows. At 24 hours, lungs are lavaged with normal saline, and alveolar protein levels in lavage fluid are determined by Lowry assay. The amount of surfactant lipids recovered by alveolar wash are determined by measuring saturated phosphatidylcholine (Sat PC) levels. Briefly, Sat PC levels are measured by extracting alveolar wash with chloroform methanol, followed by treatment of the lipid extract with OsO₄ in carbon tetrachloride and silica column chromatography. To measure cellular infiltrate the alveolar wash are centrifuged to pellet cells, and erythrocytes are lysed by hypotonic shock. Cells are resuspended, and total cell counts are determined using a hemocytometer. Differential cell counts are determined by cytocentrifugation of lavage fluid and staining with Wright stain. By use of this method, a statistically significant difference in alveolar protein levels, Sat PC levels, or neutrophil numbers is observed between the two groups, with higher levels observed in Sftpd^{-/-} mice.

### Example 33

### Generation of Pulmonary SP-D in SFTPD^{-/-} Mice for Studying for the Relative Significance of Systemic SP-D in the Treatment of Acute Respiratory Distress Syndrome (ARDS)

Sftpd^{-/-} mice expressing a doxycyline-inducible, lung specific Sftpd transgene (i.e. SP-C-rtTA/(tetO)₇-SP-D/Sftpd^{-/-} or CCSP-rtTA/(tetO)₇-SP-D/Sftpd^{-/-}) are generated (Zhang, L. et al., (2002) J Biol Chem 277:38709-38713). The SP-C and CCSP promoters are activated exclusively in the lung, and the (tetO)₇-SP-D construct places SP-D expression under the control of doxycyline induction. Pulmonary abnormalities observed in Sftpd^{-/-} mice are completely reversed by the expression of these lung specific transgenes. Therefore, these transgenic mice allow the elimination of systemic expression of SP-D, providing a means of comparing the relative significance of pulmonary versus systemic sources of SP-D in systemic immunity.

### Example 34

### Systemic SP-D is Involved in the Improvement of Symptoms of Acute Respiratory Distress Syndrome (ARDS)

Sftpd^{-/-} mice expressing a doxycyline-inducible, lung specific Sftpd transgene (Example 33) have normal levels of pulmonary SP-D and normal pulmonary morphology and alveolar macrophage function but lack all sources of systemic SP-D. To separate the relative significance of pulmonary versus systemic SP-D on ARDS in CLP mice, the markers of ARDS in Sftpd^{-/-} mice expressing a doxycyline-inducible, lung specific Sftpd transgene (Example 33) are measured and compared to ARDS marker levels in wild type and SftpdA mice. All mice are treated with doxycycline to compensate for the antimicrobial effect of doxycycline. As described in Example 27, CLP is induced in wild type, Sftpd^{-/-} and Sftpd^{-/-} mice expressing a doxycyline-inducible, lung specific Sftpd transgene. Markers of ARDS including, but not limited to, alveolar protein levels, Sat PC levels, or neutrophil infiltrate are measured as described in Example 32 and compared in tissues obtained from the three experimental mouse groups. By use of this method, a statistically significant increase in alveolar protein levels, Sat PC levels, or neutrophil numbers is observed in Sftpd^{-/-} mice expressing a doxycyline-inducible, lung specific Sftpd transgene relative to those levels found in wild type mice, indicating that systemic SP-D is involved in the improvement of symptoms of ARDS.

### Example 35

### Systemic Sources of SP-D Contribute to Plasma SP-D Pool Sizes in During Systemic Infection

Studies have indicated plasma SP-D levels increase significantly following CLP. Studies have also shown that pulmonary SP-D levels are equal in wild type and in Sftpd^{-/-} mice expressing a doxycyline-inducible, lung specific Sftpd transgene following CLP (pulmonary SP-D levels in Sftpd^{-/-} mice expressing a doxycyline-inducible, lung specific Sftpd transgene are generally higher at baseline). Therefore, if pulmonary sources of SP-D are the only source of increased plasma SP-D levels following CLP in both experimental groups, this contribution is expected to depend entirely on pulmonary leak.

Sepsis is induced in wild type and in Sftpd^{-/-} mice expressing a doxycyline-inducible, lung specific Sftpd transgene (Example 33) by subjecting them to CLP with a 30-gauge needle using the techniques as described in Example 27. Blood is collected at 48 hours, and plasma SP-D levels are determined by SP-D ELISA. By use of this method, a statistically significant decrease in plasma SP-D levels is observed in Sftpd^{-/-} mice expressing a doxycyline-inducible, lung specific Sftpd transgene relative to those levels found in wild type mice, indicating that systemic sources of SP-D contribute to plasma SP-D pool sizes during sepsis.

### Example 36

### Plasma Half-Life of SP-D Increases During Systemic Infection

Septic Sftpd^{-/-} mice are generated by CLP with a 30-gauge needle using the techniques as described in Example 27. Control Sftpd^{-/-} mice are generated by sham CLP (i.e. by exteriorizing the cecum without ligation or puncture as described in Example 27). After 48 hours, mice are administered SP-D (150 μg/kg) via tail vein injection. Blood is collected at 0.5, 1, 2, 4, 8, and 24 hours, and plasma SP-D levels are measured by SP-D ELISA. The plasma SP-D half life is then calculated. By use of this method, a statistically significant increase in plasma SP-D levels is observed in CLP-treated mice relative to control mice, indicating that the physiological mechanism used to raise plasma SP-D levels in mice is via a decrease in plasma SP-D degradation.

### Example 37

### Identification of Transcriptional Mechanisms that Control SFTPD Promoter Activity

Deletion constructs of the Sftpd promoter are used to identify regions of the promoter that are important for expression in the MFLM-91U vascular endothelial cell line. Luciferase reporter genes linked to the proximal 82, 167, 246, 357, 600, and 680 base pairs of the Sftpd gene (FIG. 21) are transfected into MFLM cells using a standard transfection protocol. Appropriate controls to normalize the amounts of transfected DNA and for efficiency of transfection are included. Luciferase activity is normalized to β-galactosidase activity using a pCMV-β-galactosidase construct. Transcription factors including, but not limited to, E-box, Nfl-like, and Pea3, which regulate gene expression in vascular endothelial cells, can be identified in the deletion analysis that correspond to consensus binding sites on the Sfptd promoter (Kou, R. et al., (2005) Biochemistry 44:15064-15073; Ardekani, A. M. et al., (1998) Thromb Haemost 80:488-494; Cieslik, K. et al., (1998) J Biol Chem 273:14885-14890, each of which is incorporated herein by reference in its entirety). One of skill in the art is also able to identify other transcription factors that can regulate systemic Sftpd expression based on sequence analysis of the Sftpd gene.

Regions of the Sftpd promoter identified by deletion analysis are further narrowed by standard DNAse I protection assays. DNAse I footprint analysis with nuclear extracts from MFLM cells and mouse lung epithelial cells (MLE-15) is conducted to define protected or hypersensitive regions of the Sftpd promoter that are specific to vascular endothelial cells. Segments of the Sftpd promoter that are protected or made hypersensitive by nuclear extracts specifically from MFLM cells are used to identify sites of transcription factor DNA binding specific vascular endothelial cells.

Candidate transcription factors identified by deletion analysis and DNAse I protection assays are fuirther investigated by co-transfection experiments. Candidate transcription factors are inserted into pCMV expression vectors and co-transfected with a Sftpd luciferase reporter construct into MFLM cells as described above, and luciferase activity is measured. By use of this method, a statistically significant difference in luciferase activity is observed relative to baseline luciferase activity in MFLM cells co-transfected with control pCMV vectors, indicating that the candidate proteins regulate Sftpd promoter activity in vascular endothelial cells.

These findings are confirmed by repeating the co-transfection experiments with an Sftpd reporter plasmid in which the candidate transcription factor consensus binding site is mutated. The results of these experiments demonstrate that the statistically significant difference in luciferase activity previously observed in co-transfection experiments with the native Sftpd consensus binding site is no longer observed when the consensus binding site is mutated.

Finally, the cell specificity of the transcriptional mechanism defined in MFLM cells in the above experiments is assessed by comparing with other cell types (i.e. HeLa and H441 cells). By use of this method, the cell specificity of the transcriptional mechanism defined in MFLM cells is confirmed by showing that the regulation of luciferase activity is observed only in MFLM cells.

### Example 38

### LPS Increases SFTPD Promoter Activity in Vascular Endothellil Cells

MFLM cells are treated with LPS (1 μg/ml), and Sftpd promoter activity is measured as described in Example 37. By use of this method, a statistically significant increase in luciferase activity is observed in LPS treated cells relative to baseline luciferase activity in non-LPS treated cells, indicating LPS increases Sftpd promoter activity in vascular endothelial cells.

### Example 39

### Systemic SP-D is Cleared by a Specific Cell Type within the Spleen

Sftpd^{-/-} mice are administered with control buffer, SP-D (200 μg/kg), or SP-D (200 μg/kg) with LPS (50 μg/kg) via tail vein injection as described in Example 19. Spleens are harvested 8 hours after injection, fixed in paraformaldehyde, embedded in paraffin and sectioned. Sections are deparafinized, rehydrated and incubated with SP-D antibody. Antibody complexes are detected using standard detection techniques (e.g. avidin-biotin-peroxidase (Vectastain), fluorescent labeling). By use of this method, cellular trafficking by specific cells in the spleen is identified.

To determine if uptake of systemic SP-D by the spleen requires the collagen domain of SP-D, these experiments are repeated with a mutant protein, rSftpdCDM, which lacks the SP-D collagen domain. By use of this method, rSftpdCDM is tracked through different tissue or cellular pathways than the full length protein, indicating that the SP-D collagen domain is important for routing and processing of SP-D in the spleen.

### Example 40

### Determination of the Mechanism by which LPS Increases SFTPD Promoter Activity in Vascular Endothelial Cells

The analysis as described in Example 37 is carried out in MFLM cells treated with LPS. Deletion constructs are tested in MFLM cells treated with LPS. Regions that are important for increasing Sftpd expression in response to LPS are analyzed by DNAse I protection assays. Comparisons between protected and hypersensitive areas observed with nuclear extracts from MFLM cells treated with control buffer versus those treated with LPS are carried out to further isolate the regions important for LPS-induced Sftpd expression in vascular endothelial cells. Candidate transcription factors are tested by cotransfection experiments and mutation of the candidate transcription factor binding site. By use of this method, a statistically significant difference in luciferase activity is observed in LPS-treated MFLM cells relative to baseline luciferase activity in non-treated MFLM cells, indicating the identity of candidate proteins that regulate LPS-induced Sftpd promoter activity in vascular endothelial cells.

### Example 41

### The SP-D Structural Features and Mechanisms Involved in Inhibiting Systemic Infections are Similar to those Used in Response to Viral Challenge in the Lung

The SP-D collagen deletion mutant, rSftpdCDM, binds bacteria and facilitates a normal response to pulmonary challenge with influenza A virus, but it fails to regulate baseline alveolar macrophage activity (i.e. macrophage activity in the absence of overt infectious challenge) or correct surfactant lipid abnormalities in Sftpd^{-/-} mice (Kingma, P. S. et al., (2006) J Biol Chem 281:24496-24505). This protein is used in experiments where separation of SP-D regulatory activity in the absence of infection from SP-D function during infectious challenge is required.

C57BL/6 mice are treated with LPS (5 μg/kg) with control buffer, SP-D (150 μg/kg), or purified rSftpdCDM (75 μg/kg, which represents an equivalent molar amount to 150 μg/kg SP-D) via tail vein injection as described in Example 19. Blood is collected 2 hours after injection, and plasma IL-6 and TNFα levels are measured by ELISA. By use of this method, it is demonstrated that rSftpdCDM inhibits systemic LPS-induced inflammation, indicating that the SP-D structural features and mechanisms used to inhibit systemic LPS-induced inflammation are similar to those utilized during viral challenge in the lung.

### Example 42

### SP-D Oligomerization is not Required for SP-D Mediated Inhibition of LPS-Induced Systemic Inflammation

SP-D is assembled predominantly as a dodecamer that is stabilized by disulfide linkages at cysteine residues 15 and 20 within the N-terminal domain. Mutant SP-D lacking these residues (rSP-DSer15/20) forms stable trimers that fail to form higher order multimers (Zhang, L. et al., (2001) J Biol Chem 276:19214-19219, which is incorporated herein by reference in its entirety). Although rSP-DSer15/20 binds carbohydrates, it fails to correct the abnormal macrophage activity in Sftpd^{-/-} mice, demonstrating the importance of SP-D oligomerization in pulmonary SP-D function.

C57BL/6 mice are treated with LPS (5 μg/kg) with control buffer, SP-D (150 μg/kg), or purified rSP-DSer15/20 (150 μg/kg) via tail vein injection as described in Example 19. Blood is collected 2 hours after injection, and plasma IL-6 and TNFα levels are measured by ELISA. By use of this method, it is demonstrated that rSP-DSer15/20 inhibits systemic LPS-induced inflammation, indicating that inhibition of systemic LPS-induced inflammation by SP-D does not depend on the multimeric structure of SP-D and that the mechanism of action of systemic SP-D is far removed from mechanisms utilized by SP-D in the lung.

### Example 43

### SP-D Inhibits Systemic Inflammation in an SP-D-Specific Manner

Both SP-D and SP-A play key roles in pulmonary host defense, but mice lacking SP-A (Sftpd^{-/-}) do not develop the enlarged, foamy macrophages that are characteristic of Sftpd^{-/-} mice, indicating that SP-D regulates alveolar macrophage activity through mechanisms that are specific for SP-D (LeVine, A. M. et al., (2000) J Immunol 165:3934-3940; LeVine, A. M. et al., (1999) Am J Respir Cell Mol Biol 20:279-286; LeVine, A. M. et al., (1999) J Clin Invest 103:1015-1021; LeVine, A. M. et al., (1998) Am J Respir Cell Mol Biol 19:700-708, each of which is incorporated herein by reference in its entirety).

C57BL/6 mice are treated with LPS (5 μg/kg) with control buffer, SP-D (150 μg/kg), or SP-A (150 μg/kg) via tail vein injection using the technique described in Example 19. Blood is collected 2 hours after injection and plasma IL-6 and TNFα levels are measured by ELISA. By use of this method, it is demonstrated that SP-A does not inhibit LPS-induced systemic inflammation, indicating that the inhibition of systemic LPS-induced inflammation is specific to SP-D and not a common property of the collectin family of proteins.

### Example 44

### Prevention of Sepsis in Newborns by Systemic Administration of SP-D

A newborn human at risk for sepsis is identified. The newborn is administered SP-D systemically using a pharmaceutical formulation at 1 mg SP-D per kg body weight. The administration is performed 4 times per day. The patient is monitored continuously. By use of this method, the susceptibility of the newborn to sepsis is decreased.

### Example 45

### Treatment of Sepsis in an Infant by Systemic Administration of SP-D

An infant diagnosed with sepsis is identified. The infant is administered SP-D systemically at 4 mg SP-D per kg body weight using a pharmaceutical formulation. The administration is performed every other hour. Plasma endotoxin levels are monitored. By use of this method, the sepsis subsides and the risk of death is decreased.

### Example 46

### Treatment of Sepsis in an Infant by Systemic Administration of 30 AA Fragment of SP-D

An infant diagnosed with sepsis is identified. The infant is systemically administered a 30 amino acid peptide corresponding to a region of SP-D at 0.5 mg peptide per kg body weight using a pharmaceutical formulation. The administration is performed every hour. The patient health is monitored continuously. By use of this method, the sepsis subsides and the risk of death is decreased.

### Example 47

### Treatment of a Lung Infection to Prevent Risk of Death of Sepsis in an Individual by Systemic Administration of SP-D

An individual with a severe lung infection is identified. The individual is at risk of developing sepsis if the lung infection continues. The patient is systemically administered SP-D at 10 mg/kg using a pharmaceutical formulation, administered two times per day. Endotoxin levels in patient plasma are measured twice a day for 5 days. Patient health is monitored continuously. By use of this method, the lung infection subsides, and the risk of developing sepsis decreases.

### Example 48

### Treatment of a Lung Infection to Prevent Risk of Death of Sepsis in an Individual by Systemic Administartion of SP-D in Combination with an Antibiotic

An individual with a severe lung infection is identified. The individual is at risk of developing sepsis if the lung infection continues. The patient is systemically administered SP-D at 1 mg/kg using a pharmaceutical formulation, administered 6 times per day. The patient is also given an oral antibiotic treatment. Endotoxin levels in patient plasma are measured twice a day for 5 days. Patient health is monitored continuously. By use of this method, the lung infection subsides, and the risk of developing sepsis decreases.

It will be apparent to one skilled in the art that varying substitutions and modifications can be made to the invention disclosed herein without departing from the scope and spirit of the invention. It is recognized that various modifications are possible within the scope of the invention disclosed. Thus, it is understood that although the present invention has been specifically disclosed by preferred embodiments and optional features, modification and variation of the concepts herein disclosed can be resorted to by those skilled in the art, and that such modifications and variations are considered to be within the scope of this invention as defined by the disclosure.

Unless otherwise indicated, all numbers expressing quantities of ingredients, properties such as experimental conditions, and so forth used in the specification are to be understood as being modified in all instances by the term “about.” Accordingly, unless indicated to the contrary, the numerical parameters set forth in the specification are approximations that can vary depending upon the desired properties sought to be determined by the present invention.

What is claimed is:

1. A method for the treatment of sepsis in a patient comprising:
administering a polypeptide having at least 70% homology to an SP-D polypeptide or a carbohydrate recognition fragment thereof to a patient in an amount effective to reduce the symptoms of sepsis.

2. The method of claim 1, wherein the polypeptide has at least 95% sequence identity to said SP-D polypeptide or a carbohydrate recognition domain fragment thereof.

3. The method of claim 1, wherein the patient is a human patient.

4. The method of claim 1, wherein the polypeptide is administered by intratracheal means, by aerosolization, or systemically.

5. The method of claim 1, wherein the sepsis is derived from a bacterial infection.

6. The method of claim 5, wherein said method is effective to decrease the leakage of E. coli cells into the blood plasma.

7. The method of claim 1, wherein said method is effective to decrease the leakage of lipopolysaccharides (LPS) into the blood plasma.

8. The method of claim 1, wherein said method is effective to decrease endotoxin levels in the blood plasma.

9. The method of claim 1, wherein said method is effective to protect said patient from the systemic effects of intratracheal endotoxin.

10. The method of claim 1, wherein said method is effective to prevent systemic inflammation.

11. The method of claim 1, wherein the sepsis is derived from a lung infection.

12. The method of claim 1, wherein the polypeptide is administered in an amount from 0.50 mg to 100 mg per kg body weight.

13. The method of claim 1, wherein the polypeptide is administered in an amount from 0.50 mg to 50 mg per kg body weight.

14. The method of claim 1, wherein the polypeptide is administered in an amount from 0.50 mg to 20 mg per kg body weight.

15. The method of claim 1, wherein the polypeptide is a recombinant polypeptide.

16. The method of claim 15, wherein the polypeptide is at least 5 amino acids in length.

17. The method of claim 1, wherein said SP-D polypeptide comprises the sequence of SEQ ID NO: 2.

18. The method of claim 1, wherein said SP-D polypeptide comprises the sequence of SEQ ID NO: 3.

19. A method for preventing sepsis in a patient comprising:
administering a polypeptide having at least 70% homology to an SP-D polypeptide or a carbohydrate recognition fragment thereof to a patient in an amount effective to prevent sepsis in the patient.

20. The method of claim 19, wherein the polypeptide is a recombinant polypeptide.

21. The method of claim 19, wherein said method is effective to prevent the leakage of E. coli cells into the blood plasma.

22. The method of claim 19, wherein said method is effective to prevent the leakage of lipopolysaccharides (LPS) into the blood plasma.

23. The method of claim 19, wherein said method is effective to prevent tissue injury during systemic infection.

24. The method of claim 23, wherein the systemic inflammation is caused by release of endotoxins from the lung.

25. The method of claim 19, wherein said method is effective to prevent LPS-induced inflammation.

26. The method of claim 19, wherein the sepsis is derived from a lung infection.


SEQUENCE LISTING (3 sequences)

SEQ ID NO: 1 - DNA, 1128 bp, Homo sapiens
atgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggacgggatgggagagagggccctcggggcgagaagggggacccaggtttgccaggagctgcagggcaagcagggatgcctggacaagctggcccagttgggcccaaaggggacaatggctctgttggagaacctggaccaaagggagacactgggccaagtggacctccaggacctcccggtgtgcctggtccagctggaagagaaggtcccctggggaagcaggggaacataggacctcagggcaagccaggcccaaaaggagaagctgggcccaaaggagaagtaggtgccccaggcatgcagggctcggcaggggcaagaggcctcgcaggccctaagggagagcgaggtgtccctggtgagcgtggagtccctggaaacacaggggcagcagggtctgctggagccatgggtccccagggaagtccaggtgccaggggacccccgggattgaagggggacaaaggcattcctggagacaaaggagcaaagggagaaagtgggcttccagatgttgcttctctgaggcagcaggttgaggccttacagggacaagtacagcacctccaggctgctttctctcagtataagaaagttgagctcttcccaaatggccaaagtgtcggggagaagattttcaagacagcaggctttgtaaaaccatttacggaggcacagctgctgtgcacacaggctggtggacagttggcctctccacgctctgccgctgagaatgccgccttgcaacagctggtcgtagctaagaacgaggctgctttcctgagcatgactgattccaagacagagggcaagttcacctaccccacaggagagtccctggtctattccaactgggccccaggggagcccaacgatgatggcgggtcagaggactgtgtggagatcttcaccaatggcaagtggaatgacagggcttgtggagaaaagcgtcttgtggtctgcgagttctga

SEQ ID NO: 2 - protein, 354 aa, Homo sapiens
AEMKTYSHRTMPSACTLVMCSSVESGLPGRDGRDGREGPRGEKGDPGLPGAAGQAGMPGQGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 3 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF